(19) United States
(12) Patent Application Publication
(10) Pub. No.: US 2023/0116871 A1
(43) Pub. Date: Apr. 13, 2023

(54) INTERBODY SPACER WITH INTEGRATED ATRAUMATIC INSERTION TOOL SYSTEM AND METHOD

(71) Applicant: Peter Ditzhazy, Spokane, WA (US)

(72) Inventor: Peter Ditzhazy, Spokane, WA (US)

(21) Appl. No.: 17/958,361

(22) Filed: Oct. 1, 2022

Related U.S. Application Data
(60) Provisional application No. 63/255,404, filed on Oct. 13, 2021.

(51) Int. Cl. A61F 2/46; A61F 2/44

(52) U.S. Cl. CPC A61F 2/4601; A61F 2/4611; A61F 2/447

(57) ABSTRACT

Systems and methods can involve (I) an interbody spacer including: (A) at least one side, and (B) at least one cavity for containing a bone graft material; and (II) an insertion tool, in which the at least one cavity is inaccessible from the at least one side when the insertion tool is engaged with the interbody spacer, and in which the at least one cavity is accessible from the at least one side when the insertion tool is disengaged from the interbody spacer. In addition, other aspects are described in the claims, drawings, and text forming a part of the present disclosure.

### BACKGROUND

With interbody fusion, first a complete discectomy and preparation of endplates of a desired disc space is performed, followed by placement of a structural implant, such as cage, spacer, or allograft, within such desired disc space. Although originating with anterior approaches to the lumbar spine, to be later refined with unilateral posterior approaches to the anterior column, more recently, lateral approaches, also known as lateral interbody fusion (LIF) has become preferred by a number of surgeons. Additional placement of bone grafts or extenders to assist interbody fusion can be performed. Furthermore, lateral interbody fusion (LIF) places the graft anterior to the instantaneous axis of rotation with the graft being exposed to compressive rather than tensile forces to likely enhance bone fusion. Also, lateral interbody fusion (LIF) can provide additional biomechanical support so that normal physiologic loads will be less likely to exceed stiffness and bending strength of posterior pedicle screw constructs. With immediate segmental stability, posterior segmental instrumentation can result in unloading and endurance limit of construct can be increased.

Lateral interbody fusion (LIF) is being used to address similar conditions that were subject of prior approaches, such as instability, deformity, infection, tumor, trauma, and degenerative disc disease. Similar to prior approaches, lateral interbody fusion (LIF) seeks to provide such results as solid fusion and restoration including (1) coronal and sagittal balance, (2) foraminal dimensions, and (3) disc space height. Unlike prior approaches, lateral interbody fusion (LIF) does so with typically minimal problems associated with prior approaches that tend to incur more complication and patient morbidity.

With its lateral approach to interbody fusion, lateral interbody fusion (LIF) involves access to the disc space through a minimally disruptive lateral, retroperitoneal, trans-psoas approach to the spine. A series of dilators are used for blunt dissection of the psoas major muscle. Since lumbar plexus nerves lie within the psoas, real-time electromyographic (EMG) monitoring directs passage. Some helpful consequences of this approach can include an absence of a requirement for mobilization of great vessels, or abdominal contents; and also, avoidance of injury to the hypogastric sympathetic plexus and avoidance of injury to the gastrointestional and genitourinary systems can occur to reduce or eliminate need for an approach surgeon. Other helpful consequences can include (1) avoidance of typical posterior approach-related complications, (2) avoidance of extensive muscle stripping and denervation that can otherwise be performed, (3) avoidance Retraction of the neural elements, and (4) avoidance of neurologic and dural related complications. Other advantages of lateral interbody fusion (LIF) can include the approach (1) being relatively simple to perform, with a rather gradual learning curve, and (2) involving decreased operative time, minimal soft-tissue disruption with less postoperative pain, minimal blood loss, shorter hospital stays and reduced time to recovery.

Lateral interbody fusion (LIF) can use relatively large interbody implants to be placed, which can more effectively restore foraminal dimensions, as well as, sagittal and coronal alignments. In optimal applications, the disc space is completely spanned by the implant from medial to lateral, which rests on the peripheral portion of the endplate, or the ring apophysis. It has been observed that the endplate peripheral portion can be stronger than the endplate central portion. As a consequence, larger implants can distribute endplate stresses distributed over a larger surface area, which lower bone-implant interface stresses. Consequently, greater resistance can be provided to implant subsidence.

Lateral interbody fusion (LIF) has been used to alleviate degenerative disc disease and axial low back pain, but its use is discouraged with cases involving severe central canal stenosis, significant scoliotic deformity, or moderate to severe spondylolisthesis. Use of lateral interbody fusion (LIF) further includes a variety of spinal pathologies, however, L5-S1 disc space exposure can be hindered by iliac crests limiting its use to anterior column stabilization above L5. These pathologies can include degenerative disc disease, complex spinal deformity, spondylolisthesis, thoracic or lumbar corpectomy and pathologies requiring lumbar total disc replacement. Other applications can include patients that need revision after either prior failed fusion surgery (pseudarthrosis, adjacent level disease) or revision of failed total disc replacement surgery. Lateral interbody fusion (LIF) can be used to remedy thoracic spine issues and with thoracic disectomy and corpectomy procedures so that thoracic disc herniations, thoracolumbar trauma, tumors and infections are also candidate conditions.

Lateral interbody fusion (LIF) includes five steps: 1) patient positioning; 2) retroperitoneal access; 3) transpsoas access and disc exposure; 4) discectomy and disc space preparation; and 5) interbody implant sizing and placement.

Regarding patient positioning, a radiolucent operating table is used that s capable of flexing near its midportion in which the patient is in the true lateral decubitus position with the greater trochanter positioned directly over the table break. The patient is secured to the operating room table using tape, and the table is flexed to increase the distance between the ribs and the iliac crest. The table is rotated as necessary to provide true AP and lateral images of the disc space.

Regarding retroperitoneal access, lateral interbody fusion (LIF) uses a one Or a two-incision technique including a direct lateral incision being the working portal centered over the target disc space. A posterolateral incision is used to gain access to retroperitoneal space and to guide safe passage of dilators and retractor system through retroperitoneal space. Skin and subcutaneous tissue are incised and abdominal obliques are bluntly dissected. Then after fascia is incised, retroperitoneal space is entered.

Regarding transpsoas access and disc exposure, after lateral interbody fusion (LIF) dilators are properly positioned and configured, the retractor system is secured and expanded, but not past midportion of vertebral body.

Regarding discectomy and end plate preparation, besides details similar to other types of spinal procedures, a lateral annulotomy is typically performed followed by a complete discectomy using pituitary rongeurs and curettes. Of note, over-aggressive decortication of the endplates should be avoided to minimize the risk of graft subsidence. Then the contralateral annulus can be released by passing a Cobb elevator completely across the disc space with both anterior annulus and posterior annulus being preserved.

Regarding interbody implant sizing and placement, proper interbody implant spacer sizing is determined, which is then packed with bone graft material. The implant is then impacted completely across the anterior to middle one-third of the disc space to position the interbody spacer over end plate outer rim on each side to provide support due to strength of the ring apophysis. Then Hemostasis is achieved, and wounds are irrigated and closed in layers.

Conventional lateral interbody fusion (LIF) procedure does have certain limitations such as it cannot be used to treat pathology involving the L5-S1 intervertebral disc since exposure is limited by the ipsilateral iliac crest.

### SUMMARY

In one or more aspects, a system for bone graft material can include (I) an interbody spacer including: (A) at least one side, and (B) at least one cavity for containing the bone graft material, and; (II) an insertion tool, wherein the at least one cavity being inaccessible from the at least one side when the insertion tool is engaged with the interbody spacer, and wherein the at least one cavity being accessible from the at least one side when the insertion tool is disengaged from the interbody spacer. Wherein (A) the at least one side of the interbody spacer includes at least one depression, and (B) the insertion tool includes at least one shield sized to block accessibility to the at least one cavity from the at least one side of the interbody spacer when the insertion tool is engaged with the interbody spacer, and (C) the insertion tool being slideably engageable and slideably disengageable with the at least one depression of the interbody spacer. Wherein the at least one depression of the interbody spacer being sized and shaped to receive the at least one shield of the insertion tool when the at least insertion tool is engaged with the interbody spacer. Wherein the at least one depression includes at least one sunken planar surface. Wherein the at least one shield of the insertion tool includes at least one plate member, the at least one plate member being slideably engageable and slideably disengageable with the at least one depression of the interbody spacer. Wherein the at least one plate member of the insertion tool being generally rectangular in shape. Wherein the at least one plate member of the insertion tool including a thickness of less than 3 mm. Wherein the at least one plate member of the insertion tool being fabricated from stainless steel. Wherein (A) the at least one depression of the interbody spacer includes at least one channel, (B) the at least one plate member of the insertion tool includes a planar surface and at least one side extending from the planar surface, and (C) the at least one channel of the interbody spacer being sized to receive the at least one side of the at least one plate member when the planar surface of the at least one plate member is in contact with the interbody spacer. Wherein the at least one channel includes a beveled open end. Wherein the at least one channel includes a first channel and a second channel having the at least one cavity positioned therebetween. Wherein (A) the at least one side of the interbody spacer includes (1) at least one planar surface portion, and (2) an edge surface extending perpendicularly to the at least one planar surface portion, (B) the at least one plate member of the insertion tool includes an end, and (C) the edge surface of the interbody spacer being in contact with the end of the at least one plate member when the insertion tool is engaged with the interbody spacer. Wherein the at least one channel includes an end, the end being adjacent to the edge surface. Wherein the at least one side includes a lip member extending from the edge surface, the lip member being parallel with the at least one planar surface portion and spaced from the at least one planar surface portion to form a gap therebetween. Wherein (A) the at least one depression of the interbody spacer includes at least one channel, (B) the at least one plate member of the insertion tool includes a planar surface and at least one side extending from a portion of the planar surface located other than on an edge of the planar surface, and (C) the at least one channel of the interbody spacer being sized to receive the at least one side of the at least one plate member when the planar surface of the at least one plate member is in contact with the interbody spacer. Wherein the insertion tool includes (A) a support member, (B) at least one plate member having an exterior surface extending in a first direction away from the support member, (C) an elongated member having a first portion and a second portion, the first portion extending from the support member, the second portion extending from the first portion in a second direction opposite the first direction, and (D) the support member includes a rear surface, the first portion of the elongated member extending other than perpendicularly from the rear surface of the support member. Wherein the insertion tool includes (A) a support member, (B) at least one plate member having an exterior surface extending in a first direction away from the support member, (C) an elongated member having a first portion and a second portion, the first portion extending from the support member, the second portion extending from the first portion in a second direction opposite the first direction, and (D) the support member includes a rear surface, the first portion of the elongated member extending from the rear surface of the support member other than parallel with the first and second directions. Wherein the insertion tool includes (A) a support member, (B) at least one plate member having an exterior surface extending in a first direction away from the support member, (C) an elongated member having a first portion and a second portion, the first portion extending from the support member, the second portion extending from the first portion in a second direction opposite the first direction, and (D) the second portion of the elongated member including an upper surface being positioned in a first plane that is unoccupied by any other portion of the insertion tool, the first plane being spaced from the exterior surface of the at least one plate member a first distance.

In one or more aspects, a system can include an interbody spacer for bone graft material, the interbody spacer including: (I) at least one side including at least one sunken planar surface; and (II) at least one cavity for containing the bone graft material, the at least one cavity including an opening adjacent the at least one sunken planar surface.

In one or more aspects, a system can include an interbody spacer for bone graft material, the interbody spacer including: (I) at least one side including: (A) at least one planar surface portion, (B) at least one cavity, the at least one cavity for containing the bone graft material, (C) at least one opening into the at least one cavity, the at least one opening being adjacent the at least one planar surface portion, (D) a first channel extending into other portions of the interbody spacer from the at least one planar surface portion, and (E) a second channel extending into other portions of the interbody spacer from the at least one planar surface portion, wherein the at least one opening into the at least one cavity being positioned between the first channel and the second channel.

In one or more aspects, a system can include an insertion tool for an interbody spacer, the insertion tool including: (I) a support member; (II) an elongated member, at least a portion of the elongated member extending from the support member along a first direction; and (III) at least one plate extending from the support member along a second direction, the second direction being opposite the first direction.

In one or more aspects, a system can include An insertion tool for an interbody spacer, the insertion tool including: (I) a support member; and (II) an elongated member including a first portion and a second portion, the first portion extending from the support member, the second portion extending from the first portion, wherein the second portion extends at an angle to the first portion.

In one or more aspects, a method for positioning an insertion tool with respect to a gap between a generally planar exposed surface of a first vertebra and a generally planar exposed surface of a second vertebra, the insertion tool including at least one plate member with a generally planar exterior surface, the insertion tool containing an interbody spacer, the method can include (I) positioning the at least one plate member of the insertion tool in a first orientation wherein the at least one plate member being a first distance from the first vertebra and wherein the generally planar exterior surface of the at least one plate member being nonparallel with the generally planar exposed surface of the first vertebra and being nonparallel with the generally planar exposed surface of the second vertebra; and (II) positioning the at least one plate member of the insertion tool in a second orientation wherein the at least one plate member being a second distance from the first vertebra and wherein the generally planar exterior surface of the at least one plate member being parallel with the generally planar exposed surface of the first vertebra and being parallel with the generally planar exposed surface of the second vertebra, wherein the first distance is greater than the second distance.

In one or more aspects, a method for positioning an interbody spacer including at least one generally planar surface with respect to a gap between a generally planar exposed surface of a first vertebra and a generally planar exposed surface of a second vertebra, the method can include (I) positioning the interbody spacer in a first orientation wherein the interbody spacer being a first distance from the first vertebra and wherein the generally planar surface of the interbody spacer being nonparallel with the generally planar exposed surface of the first vertebra and being nonparallel with the generally planar exposed surface of the second vertebra; and (II) positioning the interbody spacer in a second orientation wherein the interbody spacer being a second distance from the first vertebra and wherein the generally planar surface of the interbody spacer being parallel with the generally planar exposed surface of the first vertebra and being parallel with the generally planar exposed surface of the second vertebra, wherein the first distance is greater than the second distance.

In addition to the foregoing, other aspects are described in the claims, drawings, and text forming a part of the disclosure set forth herein. Various other aspects are set forth and described in the teachings such as text (e.g., claims and/or detailed description) and/or drawings of the present disclosure. The foregoing is a summary and thus may contain simplifications, generalizations, inclusions, or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

For a more complete understanding of implementations, reference now is made to the following descriptions taken in connection with the accompanying drawings. The use of the same symbols in different drawings typically indicates similar or identical items, unless context dictates otherwise.

With reference now to the figures, shown are one or more examples of interbody spacer and insertion tool systems and methods, articles of manufacture, compositions of matter for same that may provide context, for instance, in introducing one or more processes and/or devices described herein.

FIG. 1 is a perspective view of an interbody spacer.

FIG. 2 is a side-elevational cross-sectional view of the interbody spacer of FIG. 1 taken along the cut line 2-2 shown in FIG. 1.

FIG. 3 is a side-elevational end view of the interbody spacer of FIG. 1.

FIG. 4 is a top plan view of the interbody spacer of FIG. 1.

FIG. 5 is a top plan view of an interbody spacer.

FIG. 6 is a top plan view of an interbody spacer.

FIG. 7 is a perspective view of an insertion tool.

FIG. 8 is a side-elevational cross-sectional view of the insertion tool of FIG. 7 taken along the cut line 8-8 shown in FIG. 7.

FIG. 9 is a top plan view of the insertion tool of FIG. 7.

FIG. 10 is an enlarged top plan view of a dashed-circle portion of the insertion tool of FIG. 9 labeled “10” shown in FIG. 9.

FIG. 11 is a side-elevational view of the insertion tool of FIG. 7.

FIG. 12 is an enlarged side-elevational view of a dashed-circle portion of the insertion tool of FIG. 11 labeled “12” shown in FIG. 11.

FIG. 13 is a side-elevational end view of the insertion tool spacer of FIG. 7.

FIG. 14 is a side-elevational end view of the insertion tool spacer of FIG. 7.

FIG. 15 is a perspective view of the interbody spacer of FIG. 1 and the insertion tool of FIG. 7 before engagement therewith.

FIG. 16 is a perspective view of the interbody spacer of FIG. 1 and the insertion tool of FIG. 7 as engaged therewith.

FIG. 17 is a side-elevational cross-sectional view of the insertion tool of FIG. 7 engaged with the interbody spacer of FIG. 1 taken along the cut line 17-17 shown in FIG. 16.

FIG. 18 is a side-elevational cross-sectional view of the interbody spacer of FIG. 1 engaged with the insertion tool of FIG. 7 about to be positioned into a gap between two vertebrae.

FIG. 19 is a side-elevational cross-sectional view of the interbody spacer of FIG. 1 engaged with the insertion tool of FIG. 7 starting to be positioned into a gap between two vertebrae.

FIG. 20 is a side-elevational cross-sectional view of the interbody spacer of FIG. 1 engaged with the insertion tool of FIG. 7 further proceeding to be positioned into a gap between two vertebrae.

FIG. 21 is a side-elevational cross-sectional view of the interbody spacer of FIG. 1 engaged with the insertion tool of FIG. 7 fully positioned into a gap between two vertebrae.

FIG. 22 is a side-elevational cross-sectional view of the interbody spacer of FIG. 1 fully positioned into a gap between two vertebrae and the insertion tool of FIG. 7 being disengaged and retracted therefrom.

FIG. 23 is an enlarged side-elevational cross-sectional view of a dashed-circle portion of the interbody spacer of FIG. 1 positioned in a gap between two vertebrae of FIG. 22 labeled “23” shown in FIG. 22.

FIG. 24 is a perspective view of an interbody spacer.

FIG. 25 is a side-elevational cross-sectional view of the interbody spacer of FIG. 24 taken along the cut line 25-25 shown in FIG. 24.

FIG. 26 is a side-elevational end view of the interbody spacer of FIG. 24.

FIG. 27 is a top plan view of the interbody spacer of FIG. 24.

FIG. 28 is a perspective view of an insertion tool.

FIG. 29 is a side-elevational cross-sectional view of the insertion tool of FIG. 28 taken along the cut line 29-29 shown in FIG. 28.

FIG. 30 is a top plan view of the insertion tool of FIG. 28.

FIG. 31 is a side-elevational cross-sectional view of the insertion tool of FIG. 28.

FIG. 32 is a side-elevational end view of the insertion tool of FIG. 28.

FIG. 33 is a side-elevational end view of the insertion tool of FIG. 28.

FIG. 34 is a perspective view of the insertion tool of FIG. 24 and the insertion tool of FIG. 28 before engagement therewith.

FIG. 35 is a perspective view of the interbody spacer of FIG. 24 and the insertion tool of FIG. 35 as engaged therewith.

FIG. 36 is a side-elevational cross-sectional view of the insertion tool of FIG. 28 engaged with the interbody spacer of FIG. 24 taken along the cut line 36-36 shown in FIG. 35.

FIG. 37 is a side-elevational cross-sectional view of the interbody spacer of FIG. 24 engaged with the insertion tool of FIG. 28 fully positioned into a gap between two vertebrae.

FIG. 38 is a side-elevational cross-sectional view of the interbody spacer of FIG. 24 fully positioned into a gap between two vertebrae and the insertion tool of FIG. 28 being disengaged and retracted therefrom.

FIG. 39 is an enlarged side-elevational cross-sectional view of a dashed-circle portion of the interbody spacer positioned in a gap between two vertebrae of FIG. 38 labeled “39” shown in FIG. 38.

FIG. 40 is a perspective view of an interbody spacer.

FIG. 41 is a side-elevational cross-sectional view of the interbody spacer of FIG. 40 taken along the cut line 41-41 shown in FIG. 40.

FIG. 42 is a side-elevational end view of the interbody spacer of FIG. 41.

FIG. 43 is a top plan view of the interbody spacer of FIG. 40.

FIG. 44 is a side-elevational cross-sectional view of the interbody spacer of FIG. 40 engaged with the insertion tool of FIG. 28 fully positioned into a gap between two vertebrae.

FIG. 45 is a side-elevational cross-sectional view of the interbody spacer of FIG. 40 fully positioned into a gap between two vertebrae and the insertion tool of FIG. 28 being disengaged and retracted therefrom.

FIG. 46 is an enlarged side-elevational cross-sectional view of a dashed-circle portion of the interbody spacer positioned in a gap between two vertebrae of FIG. 45 labeled “46” shown in FIG. 45.

FIG. 47 is a side-elevational end view of an insertion tool.

FIG. 48 is a side-elevational end view of the insertion tool of FIG. 47.

FIG. 48A is a cross-sectional side-elevational view of the insertion tool of FIG. 47.

FIG. 48B is a side-elevational cross-sectional view of the interbody spacer of FIG. 40 engaged with the insertion tool of FIG. 47 fully positioned into a gap between two vertebrae.

FIG. 49 is a side-elevational cross-sectional view of an insertion tool.

FIG. 50 is a side-elevational cross-sectional view of the interbody spacer of FIG. 1 engaged with the insertion tool of FIG. 49 at a first angle and commencing to be positioned into a gap between two vertebrae.

FIG. 51 is a side-elevational cross-sectional view of the interbody spacer of FIG. 1 engaged with the insertion tool of FIG. 49 at the first angle of FIG. 50 and incrementally closer to be positioned into a gap between two vertebrae.

FIG. 52 is a side-elevational cross-sectional view of the interbody spacer of FIG. 1 engaged with the insertion tool of FIG. 49 at a first angle of FIG. 50 and incrementally closer to be positioned into a gap between two vertebrae.

FIG. 53 is a side-elevational cross-sectional view of the interbody spacer of FIG. 1 engaged with the insertion tool of FIG. 49 at a second angle and incrementally closer to be positioned into a gap between two vertebrae.

FIG. 54 is a side-elevational cross-sectional view of the interbody spacer of FIG. 1 engaged with the insertion tool of FIG. 49 at the second angle of 53 and incrementally closer to be positioned into a gap between two vertebrae.

FIG. 55 is a side-elevational cross-sectional view of the interbody spacer of FIG. 1 engaged with the insertion tool of FIG. 49 at the first angle of FIG. 50 and incrementally closer to be positioned into a gap between two vertebrae.

FIG. 56 is a side-elevational cross-sectional view of the interbody spacer of FIG. 1 engaged with the insertion tool of FIG. 49 fully positioned into a gap between two vertebrae.

FIG. 57 is a side-elevational cross-sectional view of the interbody spacer of FIG. 1 fully positioned into a gap between two vertebrae and the insertion tool of FIG. 49 at the first angle of FIG. 50 being disengaged and retracted therefrom.

FIG. 58 is a side-elevational cross-sectional view of the interbody spacer of FIG. 1 fully positioned into a gap between two vertebrae and the insertion tool of FIG. 49 at the second angle of FIG. 53 being disengaged and further incrementally retracted therefrom.

FIG. 59 is a side-elevational cross-sectional view of the interbody spacer of FIG. 1 fully positioned into a gap between two vertebrae and the insertion tool of FIG. 49 at the second angle of FIG. 53 being disengaged and further incrementally retracted therefrom.

FIG. 60 is a side-elevational cross-sectional view of the interbody spacer of FIG. 1 fully positioned into a gap between two vertebrae and the insertion tool of FIG. 49 at the first angle of FIG. 50 being disengaged and further incrementally retracted therefrom.

FIG. 61 is a side-elevational cross-sectional view of the interbody spacer of FIG. 1 fully positioned into a gap between two vertebrae and the insertion tool of FIG. 49 at the first angle of FIG. 50 being disengaged and further incrementally retracted therefrom.

FIG. 62 is a side-elevational cross-sectional view of the interbody spacer of FIG. 1 fully positioned into a gap between two vertebrae and the insertion tool of FIG. 49 at the first angle of FIG. 50 being disengaged and further incrementally retracted therefrom.

FIG. 63 is a side-elevational cross-sectional view of the interbody spacer of FIG. 24 being engaged with an insertion tool.

FIG. 64 is a side-elevational cross-sectional view of the interbody spacer of FIG. 40 being engaged with the insertion tool of FIG. 63.

### DETAILED DESCRIPTION

In the following detailed description, reference is made to the accompanying drawings, which form a part hereof. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here.

Implementations of systems and methods described herein concern, in general, lumbar interbody fusion procedures and, in particular, minimally invasive spine surgery involving lateral interbody fusion (LIF) and anterior interbody fusion (ALIF) using a lateral, retroperitoneal, trans-psoas or anterior approach for desired disc space access.

The approaches described below create surface pressure/load between the vertebral end plates and the implant during insertion and repositioning. These loads in conjunction with aggressively textured traumatic implants or custom implants designed to realign the spine, can lead to end plate violation. This intern can lead to coronal or sagittal alignment issues and possible revision surgery. Furthermore approach angles when inserting the implant can be effected by several anatomical variances, (high Iliac crest or rib cage). These approach angles can create uneven load disbursement and localized or specific end plate violation and subsequent sequela.

Interbody spacers can be typically surgically placed between two vertebrae. It is envisioned that implementations described herein offer improvements in placement procedures for interbody spacers by, for instance, reducing frictional loads experienced during such procedures. Implementations also offer other enhancements such as providing for, during placement procedures, protection of materials being contained by interbody spacers while also affording increased exposure of such materials to vertebrae surfaces after placement has been accomplished. Further enhancements can allow for nonconventional orientation options during interbody spacer placement procedures to address occasional hinderances otherwise presented by configurations of patient internal body structures.

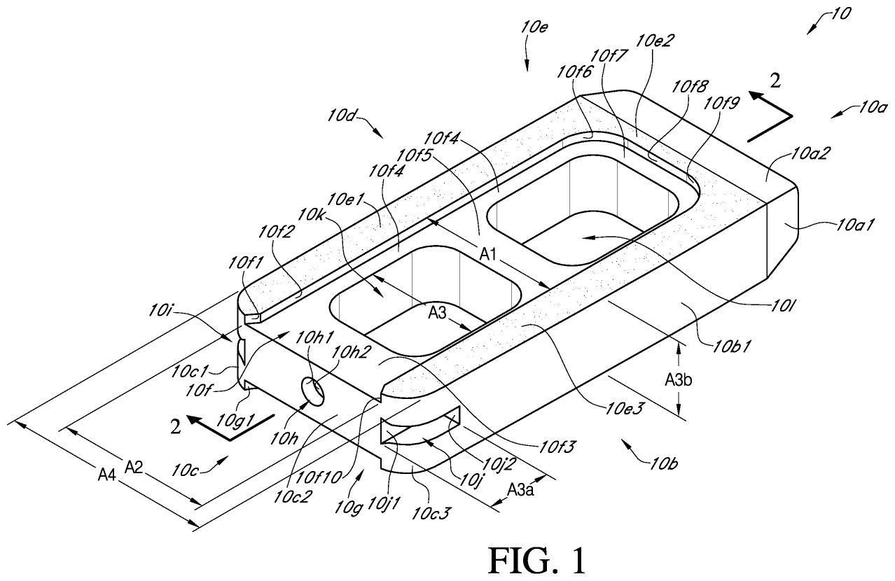

Turning to FIG. 1, depicted therein is a perspective view of interbody spacer 10. In implementations, interbody spacer 10 is shown to include distal tapered lateral end 10a, right side 10b, proximal lateral end 10c, left side 10d, upper side 10e, sunken upper portion 10f, sunken lower portion 10g, receptacle opening 10h, left slot 10i, right slot 10j, proximal cavity 10k, and distal cavity 10l. In general, interbody spacer 10 and other interbody spacers discussed herein can includes sides of vary heights either with respect to one another, or with respect to various portions of a particular side. Hence, interbody spacer 10 and other interbody spacer implementations are not limited to the depicted shapes, but can be sized and shaped according to medical constraints or other factors directed to a particular implantation procedure. Also, in other embodiments, one or more cavities can be shaped other than those depicted herein, such as proximal cavity 10k, and distal cavity 10l. These shapes can include, but are not limited to, elliptical, rectangular, circular, semi-circular, etc. Furthermore, interbody spacer 10 can be manufactured from biologically accepted inert material, such as polyether ether ketone (PEEK), other thermoplastics, radiolucent materials, alloys, metals, or other materials having desired structural, mechanical, thermal resistance, chemical, or other desired properties.

In implementations, distal tapered lateral end 10a is shown to include right surface 10al, and upper surface 10a2. In implementations, right side 10b is shown to include right surface 10b1. In implementations, proximal lateral end 10c is shown to include proximal left corner 10c1, proximal surface 10c2, and proximal right corner 10c3. In implementations, upper side 10e is shown to include left upper surface 10e1, distal upper surface 10e2, and right upper surface 10e3. In implementations, sunken upper portion 10f is shown to include left beveled edge 10f1, left edge 10f2, proximal surface portion 10f3, left surface portion 10f4, mid surface portion 10f5, left distal corner edge 10f6, distal surface portion 10f7, distal edge 10f8, right distal corner edge 10f9, and right beveled edge 10f10. In implementations, sunken lower portion 10g is shown to include left beveled edge 10g1. In implementations, receptacle opening 10h is shown to include circumferential surface 10h1, and end surface 10h2. In implementations, right slot 10j is shown to include left surface 10j1, and distal surface 10j2. In implementations, interbody spacer 10 is shown to include linear dimension A1, linear dimension A2, linear dimension A3, linear dimension A3a, linear dimension A3b, and linear dimension A4.

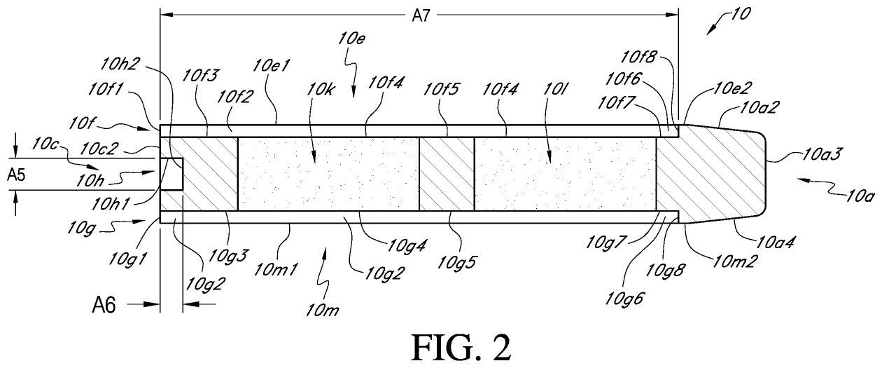

Turning to FIG. 2, depicted therein is a side-elevational cross-sectional view of interbody spacer 10 of FIG. 1 taken along cut line 2-2 shown in FIG. 1. In implementations, distal tapered lateral end 10a is shown to include distal surface 10a3, and lower surface 10a4. In implementations, sunken lower portion 10g is shown to include right edge 10g2, proximal surface portion 10g3, left surface portion 10g4, mid surface portion 10g5, left distal corner edge 10g6, distal surface portion 10g7, and distal edge 10g8. In implementations, interbody spacer 10 is shown to include lower side 10m. In implementations, lower side 10m is shown to include left lower surface 10m1, and distal lower surface 10m2. In implementations, interbody spacer 10 is shown to include linear dimension A5, linear dimension A6, and linear dimension A7.

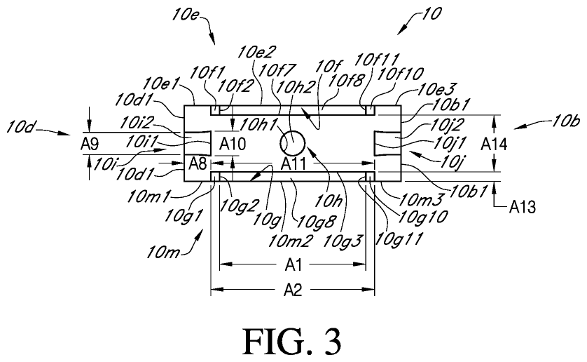

Turning to FIG. 3, depicted therein is a side-elevational end view of interbody spacer 10 of FIG. 1. In implementations, left side 10d is shown to include left surface 10d1. In implementations, sunken upper portion 10f is shown to include right edge 10f11. In implementations, sunken lower portion 10g is shown to include right beveled edge 10g10, and left edge 10g11. In implementations, left slot 10i is shown to include right surface 10i1, and distal surface 10i2. In implementations, lower side 10m is shown to include right lower surface 10m3. In implementations, interbody spacer 10 is shown to include linear dimension A8, linear dimension A9, linear dimension A10, linear dimension A11, linear dimension A13, and linear dimension A14.

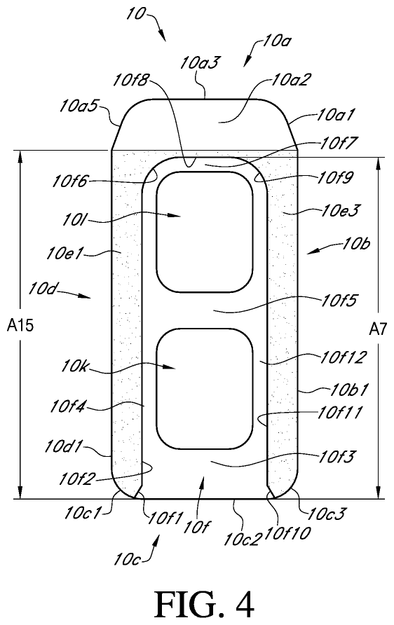

Turning to FIG. 4, depicted therein is a top plan view of interbody spacer 10 of FIG. 1. In implementations, distal tapered lateral end 10a is shown to contain left surface 10a5. In implementations, sunken upper portion 10f is shown to include right surface portion 10f12. In implementations, interbody spacer 10 is shown to include linear dimension A15.

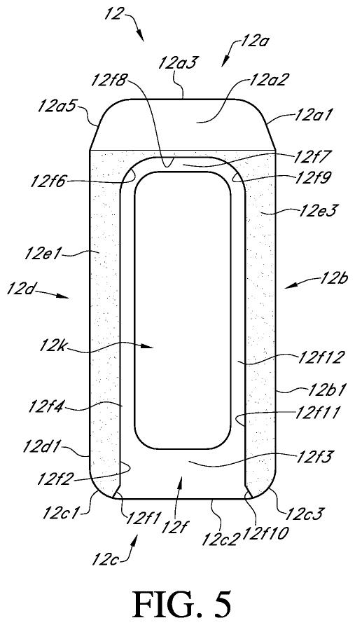

Turning to FIG. 5, depicted therein is a top plan view of interbody spacer 12. In implementations, interbody spacer 12 is shown to include distal tapered lateral end 12a, right side 12b, proximal lateral end 12c, left side 12d, sunken upper portion 12f, and cavity 12k. In implementations, distal tapered lateral end 12a is shown to include right surface 12a1, upper surface 12a2, distal surface 12a3, and left surface 12a5. In implementations, right side 12b is shown to include right surface 12b1. In implementations, proximal lateral end 12c is shown to include proximal left corner 12c1, proximal surface 12c2, and proximal right corner 12c3. In implementations, left side 12d is shown to include left surface 12d1. In implementations, interbody spacer 12 is shown to include left upper surface 12e1, and right upper surface 12e3. In implementations, sunken upper portion 12f is shown to include left beveled edge 12f1, left edge 12f2, proximal surface portion 12f3, left surface portion 12f4, left distal corner edge 12f6, distal surface portion 12f7, distal edge 12f8, right distal corner edge 12f9, right beveled edge 12f10, right edge 12f11, and right surface portion 12f12.

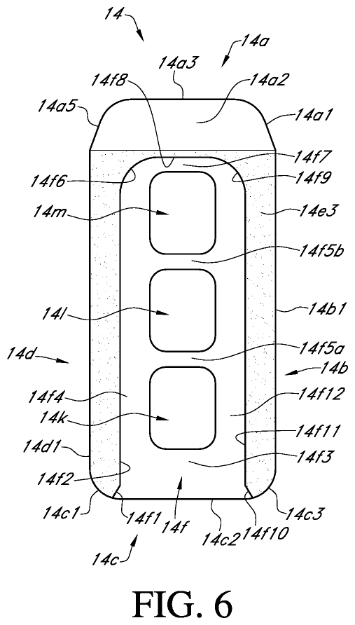

Turning to FIG. 6, depicted therein is a top plan view of interbody spacer 14. In implementations, interbody spacer 14 is shown to include distal tapered lateral end 14a, right side 14b, proximal lateral end 14c, left side 14d, right upper surface 14e3, sunken upper portion 14f, proximal cavity 14k, mid cavity 14l, and distal cavity 14m. In implementations, distal tapered lateral end 14a is shown to include right surface 14a1, upper surface 14a2, distal surface 14a3, and left surface 14a5. In implementations, right side 14b is shown to include right surface 14b1. In implementations, proximal lateral end 14c is shown to include proximal left corner 14c1, proximal surface 14c2, and proximal right corner 14c3. In implementations, left side 14d is shown to include left surface 14d1. In implementations, sunken upper portion 14f is shown to include left beveled edge 14f1, left edge 14f2, proximal surface portion 14f3, left surface portion 14f4, left distal corner edge 14f6, distal surface portion 14f7, distal edge 14f8, right distal corner edge 14f9, right beveled edge 14f10, right edge 14f11, and right surface portion 14f12.

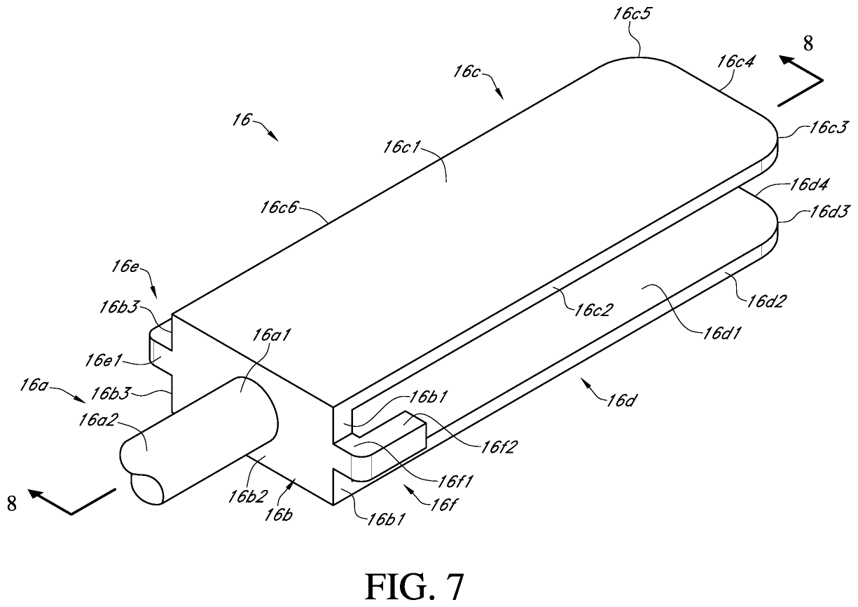

Turning to FIG. 7, depicted therein is a perspective view of insertion tool 16. In implementations, insertion tool 16 is shown to include elongated member 16a, support member 16b, upper plate member 16c, lower plate member 16d, left tongue member 16e, and right tongue member 16f. In implementations, elongated member 16a is shown to include distal portion 16a1, and proximal portion 16a2. In implementations, support member 16b is shown to include right surface 16b1, proximal surface 16b2, and left surface 16b3. In implementations, upper plate member 16c is shown to include exterior surface 16c1, right side 16c2, right distal corner 16c3, distal side 16c4, left distal corner 16c5, and left side 16c6. In implementations, lower plate member 16d is shown to include interior surface 16d1, right side 16d2, right distal corner 16d3, and distal side 16d4. In implementations, left tongue member 16e is shown to include extension portion 16e1. In implementations, right tongue member 16f is shown to include extension portion 16f1, and tongue portion 16f2.

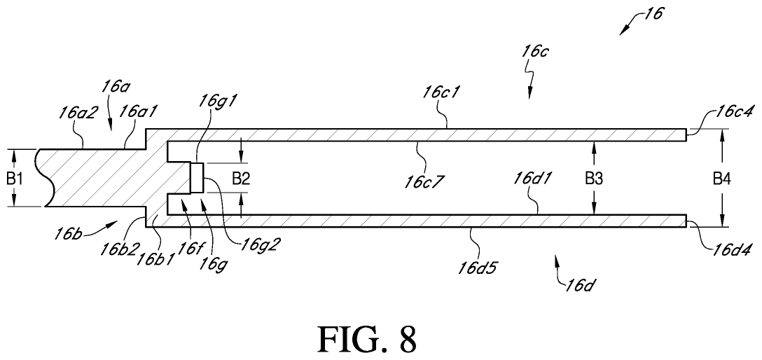

Turning to FIG. 8, depicted therein is a side-elevational cross-sectional view of insertion tool 16 of FIG. 7 taken along cut line 8-8 shown in FIG. 7. In implementations, upper plate member 16c is shown to include interior surface 16c7. In implementations, lower plate member 16d is shown to include exterior surface 16d5. In implementations, engagement member 16g is shown to include circumferential surface 16g1, and end surface 16g2. In implementations, insertion tool 16 is shown to include linear dimension B1, linear dimension B2, linear dimension B3, and linear dimension B4.

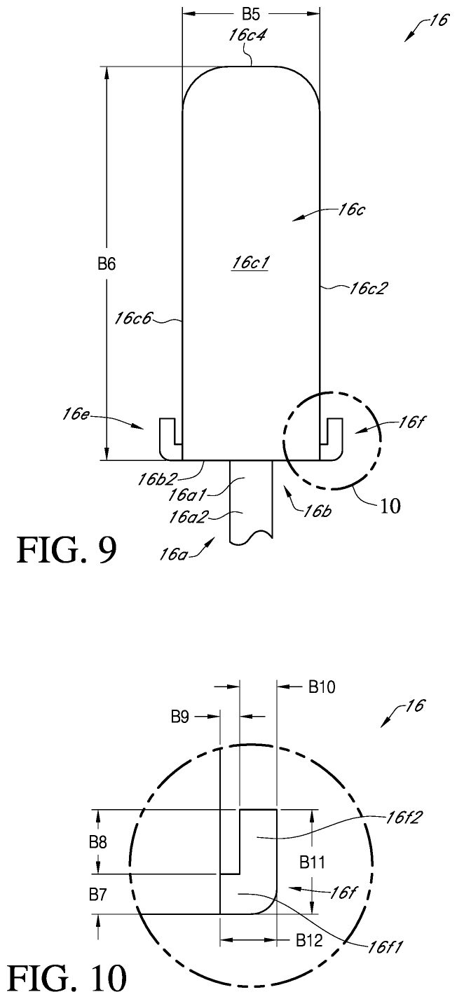

Turning to FIG. 9, depicted therein is a top plan view of insertion tool 16 of FIG. 7. In implementations, insertion tool 16 is shown to include linear dimension B5, and linear dimension B6.

Turning to FIG. 10, depicted therein is an enlarged top plan view of a dashed-circle portion of insertion tool 16 of FIG. 9 labeled “10” shown in FIG. 9. In implementations, insertion tool 16 is shown to include linear dimension B7, linear dimension B8, linear dimension B9, linear dimension B10, linear dimension B11, and linear dimension B12.

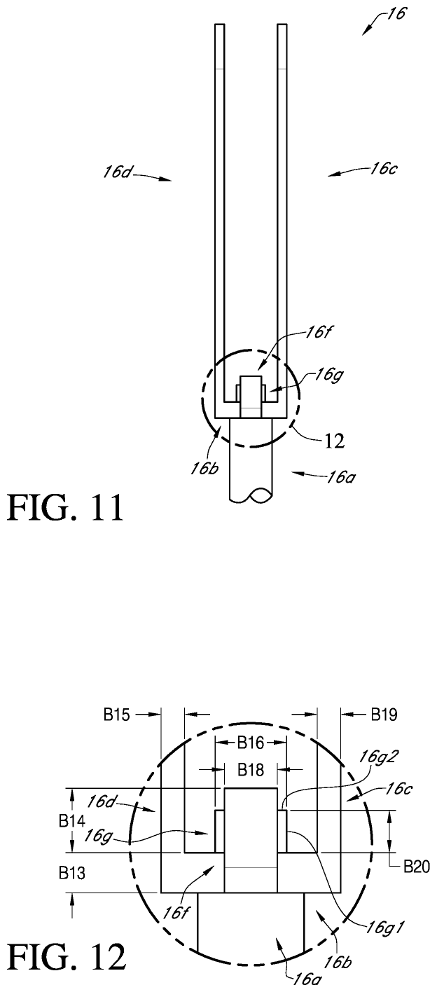

Turning to FIG. 11, depicted therein is a side-elevational view of insertion tool 16 of FIG. 7.

Turning to FIG. 12, depicted therein is an enlarged side-elevational view of a dashed-circle portion of insertion tool 16 of FIG. 11 labeled “12” shown in FIG. 11. In implementations, insertion tool 16 is shown to include linear dimension B13, linear dimension B14, linear dimension B15, linear dimension B16, linear dimension B18, linear dimension B19, and linear dimension B20. Manufacture of insertion tool 16 and other insertion tool implementations can utilize one or more rigid materials, such as, hardened stainless steel, other types or grades of steel, titanium, other metals or alloys, composites, natural or synthetic materials, etc.

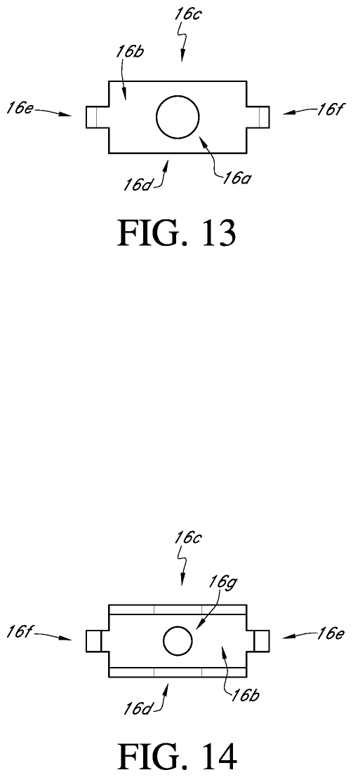

Turning to FIG. 13, depicted therein is a side-elevational end view of insertion tool 16 of FIG. 7.

Turning to FIG. 14, depicted therein is a side-elevational end view of insertion tool 16 of FIG. 7.

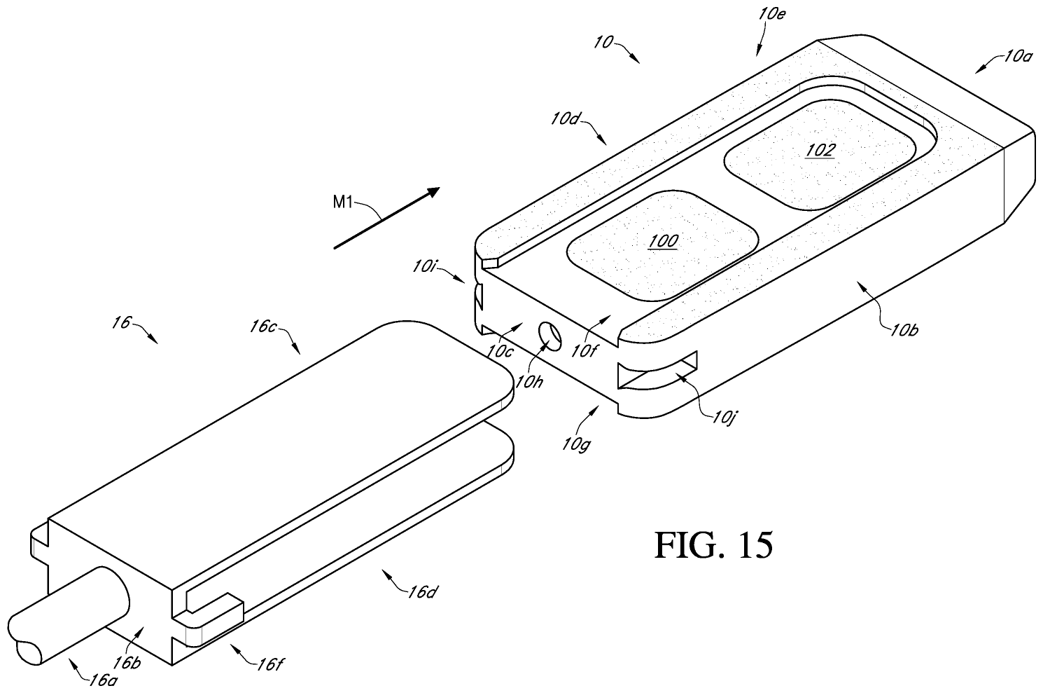

Turning to FIG. 15, depicted therein is a perspective view of interbody spacer 10 of FIG. 1 containing bone graft material 100 and bone graft material 102, and insertion tool 16 of FIG. 7 moving in direction M1 before engagement therewith. The bone graft material 100 and bone graft material 102 can include, but are not limited to, Demineralized Bone Matrix (“DBM”) packing, bone morphogenetic protein (BMP), collagen matrix, bone cement, other flowable grafting agents or materials, flaky or other non-flowable grafting agents or materials, other biological or non-biological materials or substances, or any other suitable grafting, filler, sponge, foam, or other porous or absorbent structure material.

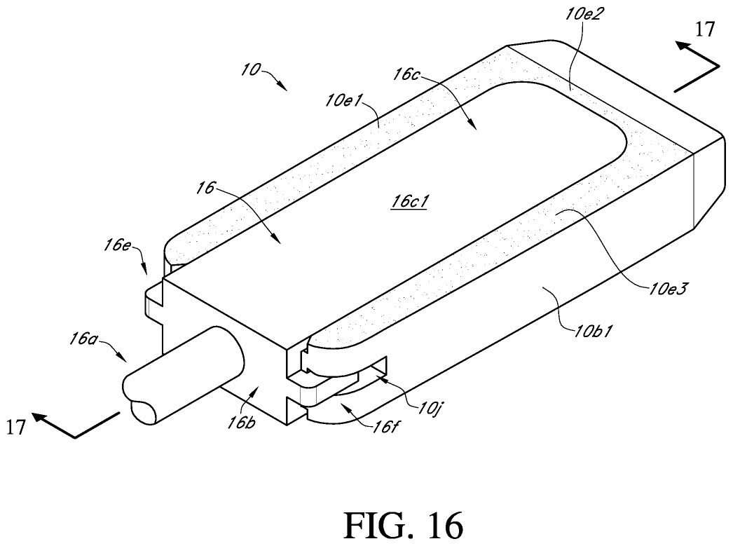

Turning to FIG. 16, depicted therein is a perspective view of interbody spacer 10 of FIG. 1 and insertion tool 16 of FIG. 7 as engaged therewith.

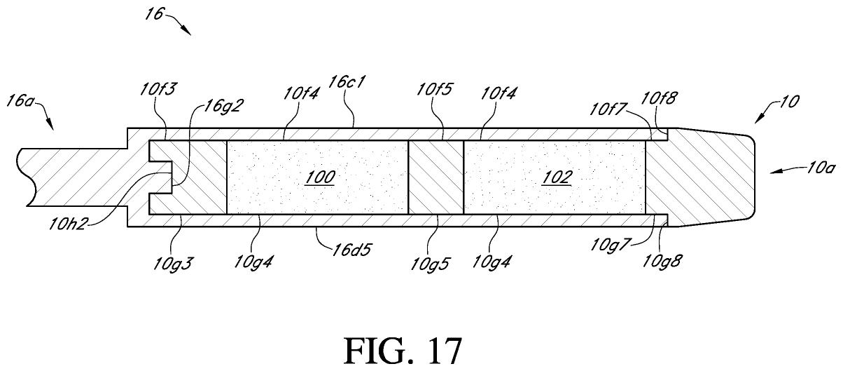

Turning to FIG. 17, depicted therein is a side-elevational cross-sectional view of insertion tool 16 of FIG. 7 engaged with interbody spacer 10 of FIG. 1 taken along cut line 17-17 shown in FIG. 16. As shown, interbody spacer 10 of interbody spacer 10 is releasably engaged with interbody spacer 10 of interbody spacer 10. Such engagement can be accomplished with cooperative threading of interbody spacer 10 to match threading of interbody spacer 10. Other engagement approaches can include utilized such as slip-fit, press-fit, friction-fit, tabbed connections, or any other standard or non-standard coupling considerations.

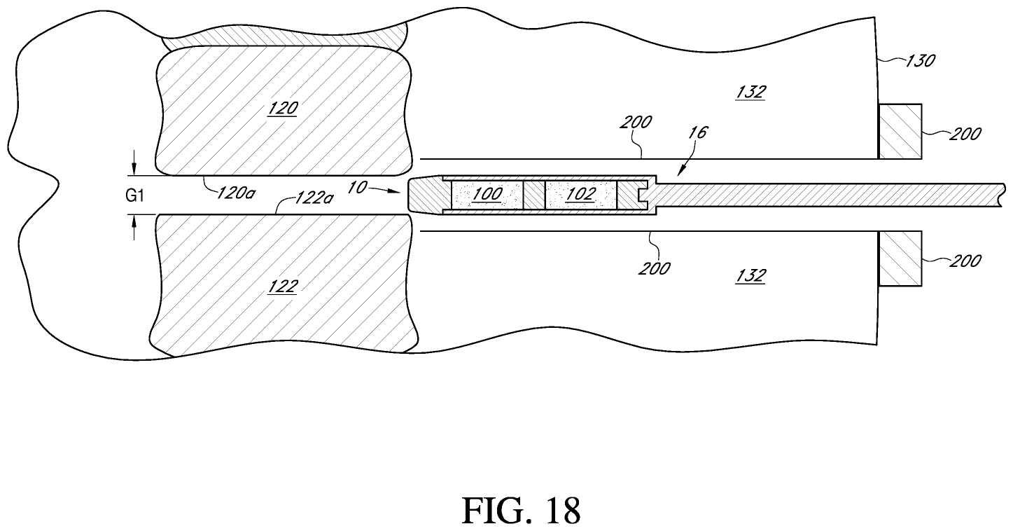

Turning to FIG. 18, depicted therein is a side-elevational cross-sectional view of interbody spacer 10 of FIG. 1 engaged with insertion tool 16 of FIG. 7 inside of retractor tool 200 that is running through body cavity 132 from body exterior 130 to gap spacing G1 between vertebra 120 and vertebra 122. As shown, interbody spacer 10 engaged with insertion tool 16 is about to be positioned into gap spacing G1 between vertebra 120 having exposed surface 120a and vertebra 122 having exposed surface 122a.

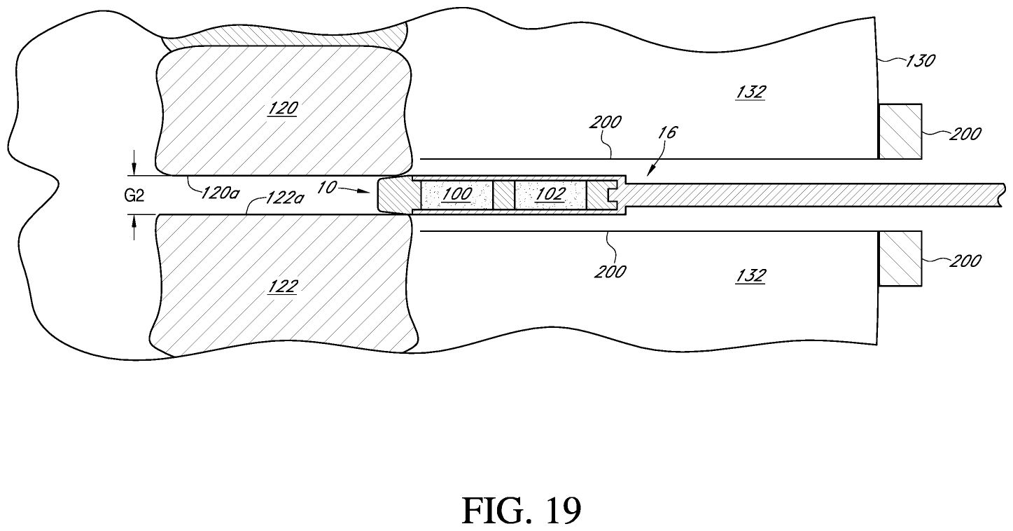

Turning to FIG. 19, depicted therein is a side-elevational cross-sectional view of interbody spacer 10 of FIG. 1 engaged with insertion tool 16 of FIG. 7 starting to be positioned into gap spacing G2 between vertebra 120 and vertebra 122.

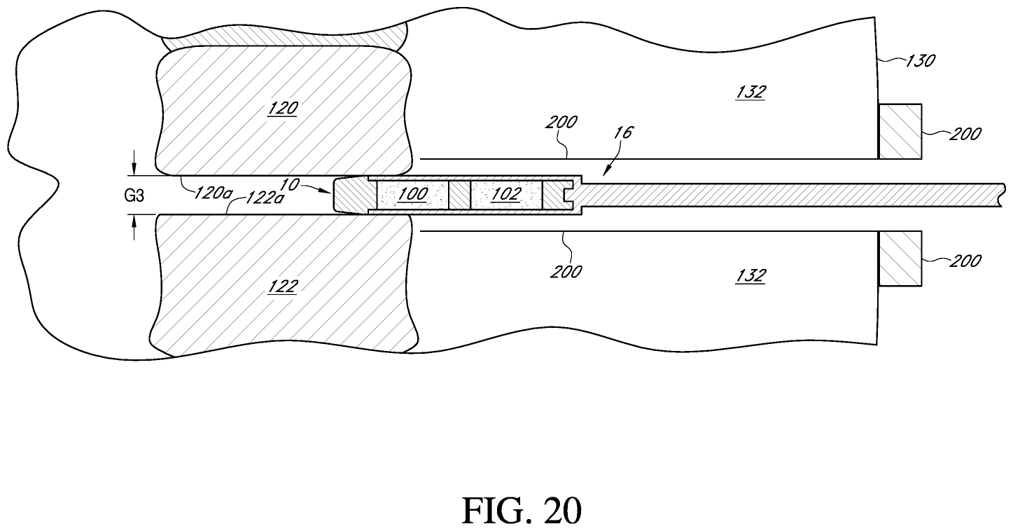

Turning to FIG. 20, depicted therein is a side-elevational cross-sectional view of interbody spacer 10 of FIG. 1 engaged with insertion tool 16 of FIG. 7 further proceeding to be positioned into gap spacing G3 between vertebra 120 and vertebra 122.

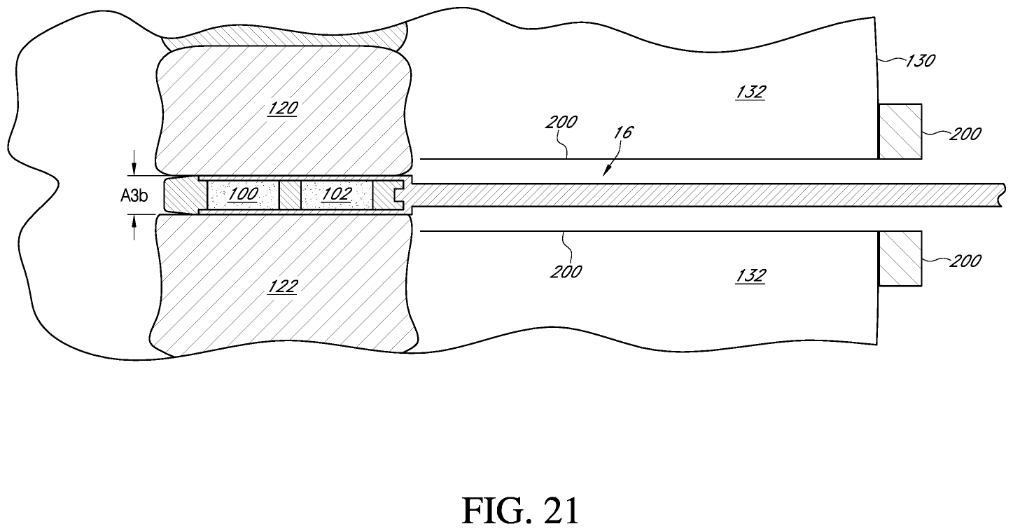

Turning to FIG. 21, depicted therein is a side-elevational cross-sectional view of interbody spacer 10 of FIG. 1 engaged with insertion tool 16 of FIG. 7 fully positioned between vertebra 120 and vertebra 122.

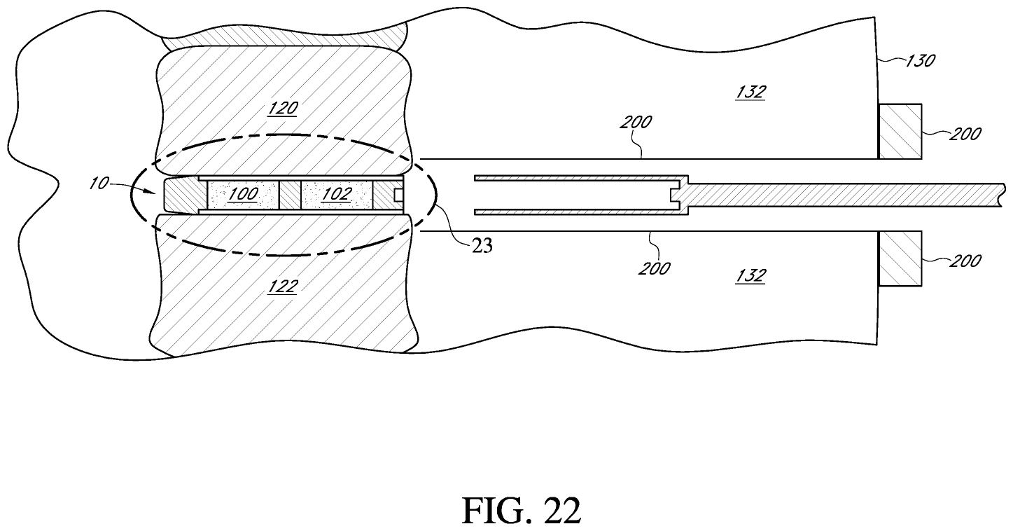

Turning to FIG. 22, depicted therein is a side-elevational cross-sectional view of interbody spacer 10 of FIG. 1 fully positioned into a gap between two vertebrae and insertion tool 16 of FIG. 7 being disengaged and retracted therefrom.

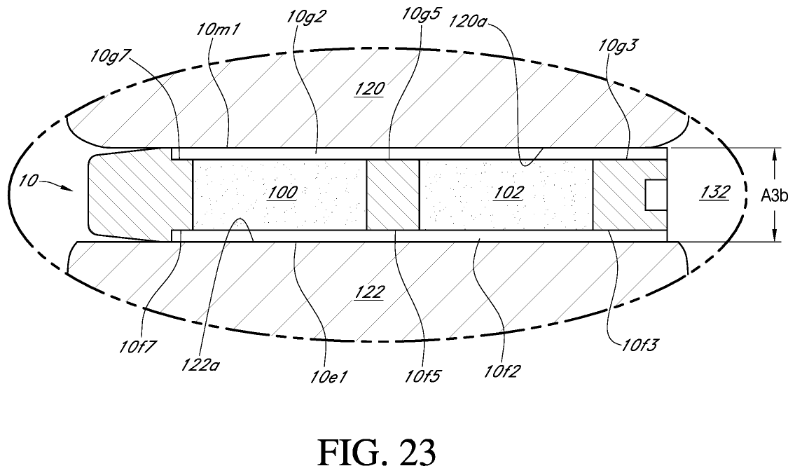

Turning to FIG. 23, depicted therein is an enlarged side-elevational cross-sectional view of a dashed-circle portion of interbody spacer 10 of FIG. 1 positioned in a gap between two vertebrae of FIG. 22 labeled “23” shown in FIG. 22.

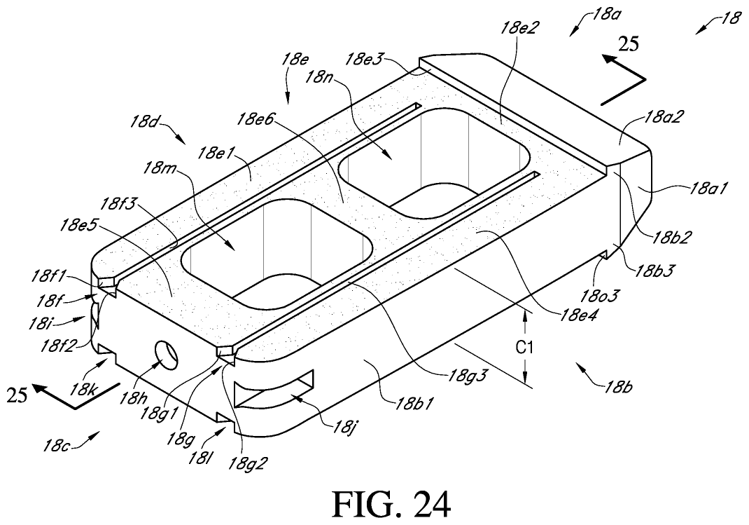

Turning to FIG. 24, depicted therein is a perspective view of interbody spacer 18. In implementations, interbody spacer 18 is shown to include distal tapered lateral end 18a, right side 18b, proximal lateral end 18c, left side 18d, upper side 18e, left upper channel 18f, right upper channel 18g, receptacle opening 18h, left groove 18i right groove 18j, left lower channel 18k, right lower channel 18l, proximal cavity 18m, distal cavity 18n, and distal lower edge 18o3. In implementations, distal tapered lateral end 18a is shown to include right surface 18a1, and upper surface 18a2. In implementations, right side 18b is shown to include right surface portion 18b1, upper distal right surface portion 18b2, and lower distal right surface portion 18b3. In implementations, upper side 18e is shown to include left upper surface 18e1, distal upper surface 18e2, distal upper edge 18e3, right upper surface 18e4, proximal upper surface 18e5, and mid upper surface 18e6. In implementations, left upper channel 18f is shown to include left beveled distal edge 18f1, right beveled distal edge 18f2, and left side 18f3. In implementations, right upper channel 18g is shown to include left beveled distal edge 18g1, right beveled distal edge 18g2, and left side 18g3.

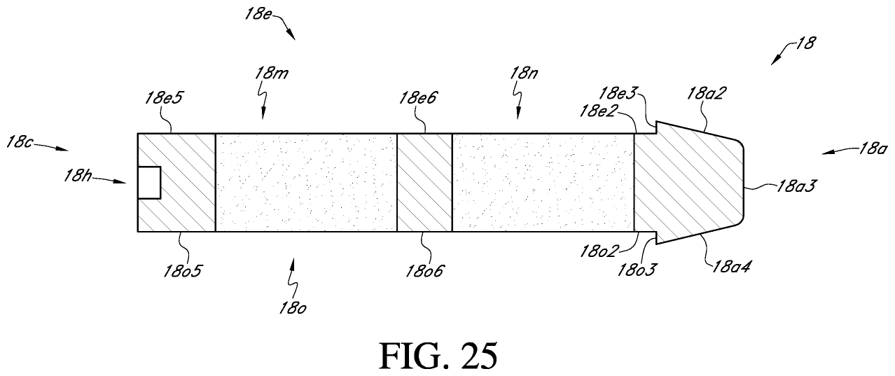

Turning to FIG. 25, depicted therein is a side-elevational cross-sectional view of interbody spacer 18 of FIG. 24 taken along cut line 25-25 shown in FIG. 24. In implementations, interbody spacer 18 is shown to include lower side 180. In implementations, distal tapered lateral end 18a is shown to include distal surface 18a3, and lower surface 18a4. In implementations, lower side 18o is shown to include distal lower surface 18o2, proximal upper surface 18o5, and mid upper surface 18o6.

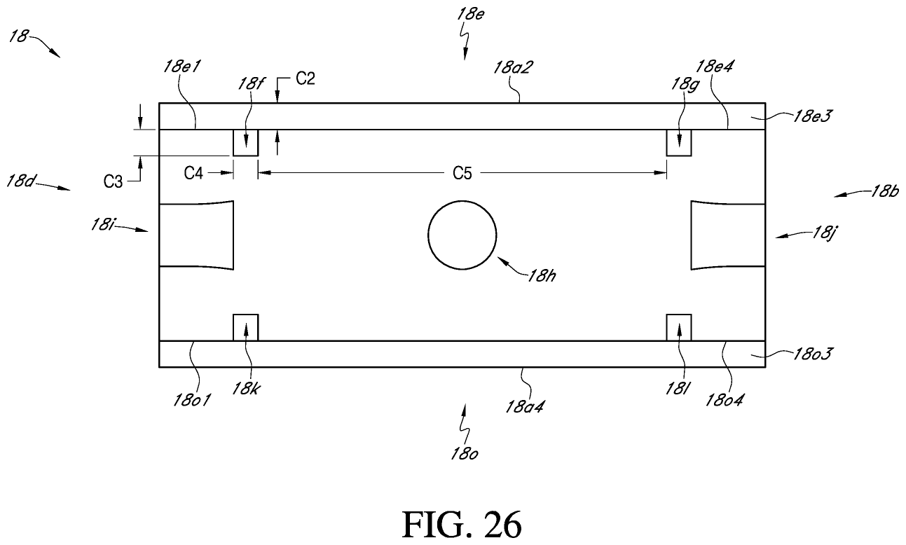

Turning to FIG. 26, depicted therein is a side-elevational end view of interbody spacer 18 of FIG. 24. In implementations, lower side 18o is shown to include left lower surface 18o1, and right lower surface 18o4. In implementations, interbody spacer 18 is shown to include linear dimension C2, linear dimension C3, linear dimension C4, and linear dimension C5.

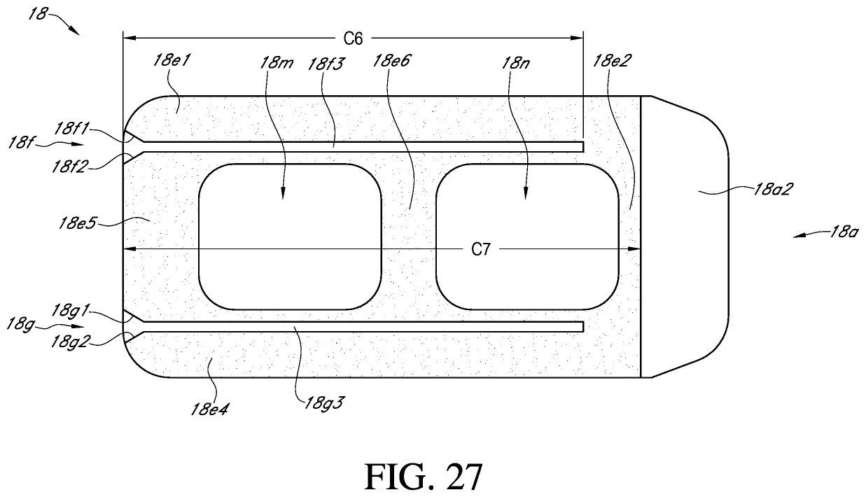

Turning to FIG. 27, depicted therein is a top plan view of interbody spacer 18 of FIG. 24. In implementations, interbody spacer 18 is shown to include linear dimension C7.

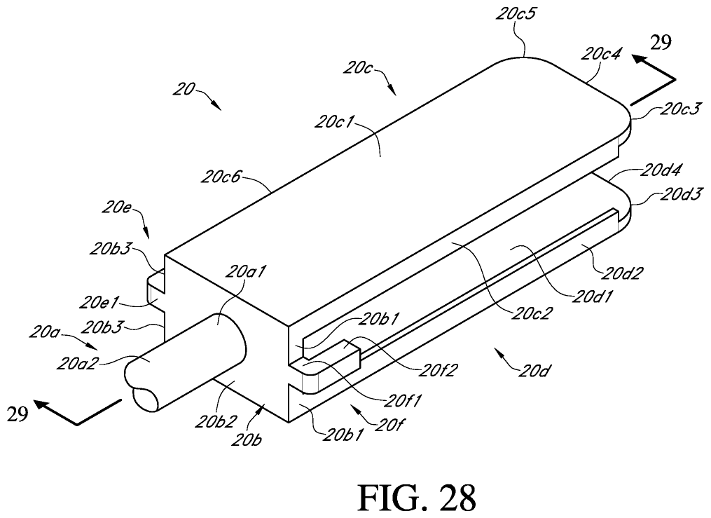

Turning to FIG. 28, depicted therein is a perspective view of an insertion tool 20. In implementations, insertion tool 20 is shown to include elongated member 20a, support member 20b, upper plate member 20c, lower plate member 20d, left tongue member 20e, and right tongue member 20f. In implementations, elongated member 20a is shown to include distal portion 20a1, and proximal portion 20a2. In implementations, support member 20b is shown to include right surface 20b1, proximal surface 20b2, and left surface 20b3. In implementations, upper plate member 20c is shown to include exterior surface 20c1, right side 20c2, right distal corner 20c3, distal side 20c4, left distal corner 20c5, and left side 20c6. In implementations, lower plate member 20d is shown to include interior surface 20d1, right side 20d2, right distal corner 20d3, and distal side 20d4. In implementations, right tongue member 20f is shown to include extension portion 20f1, and tongue portion 20f2.

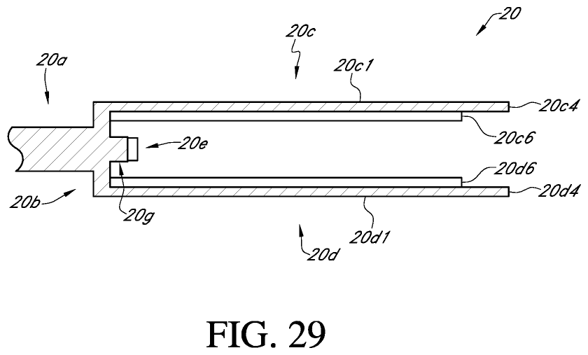

Turning to FIG. 29, depicted therein is a side-elevational cross-sectional view of insertion tool 20 of FIG. 28 taken along cut line 29-29 shown in FIG. 28. In implementations, insertion tool 20 is shown to include engagement member 20g. In implementations, lower plate member 20d is shown to include left side 20d6.

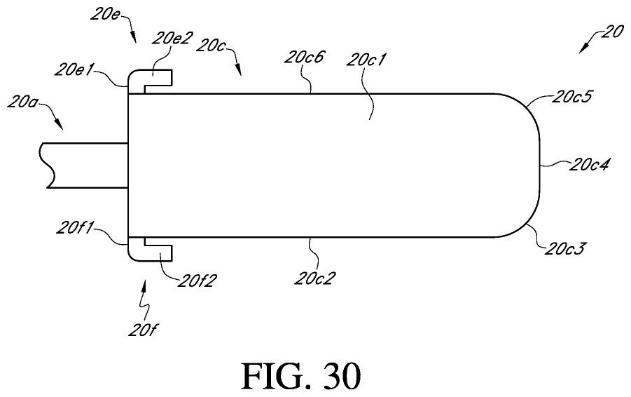

Turning to FIG. 30, depicted therein is a top plan view of insertion tool 20 of FIG. 28. In implementations, left tongue member 20e is shown to include tongue portion 20e2.

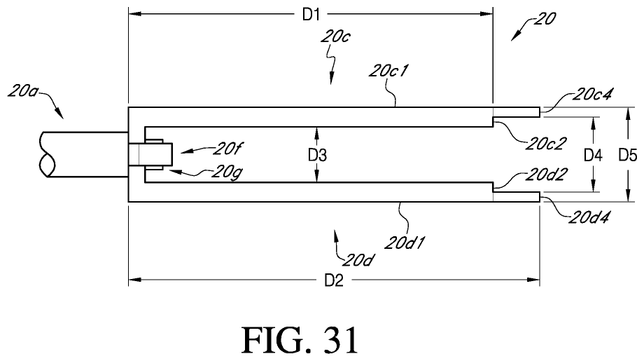

Turning to FIG. 31, depicted therein is a side-elevational cross-sectional view of insertion tool 20 of FIG. 28. In implementations, insertion tool 20 is shown to include linear dimension D1, linear dimension D2, linear dimension D3, linear dimension D4, and linear dimension D5.

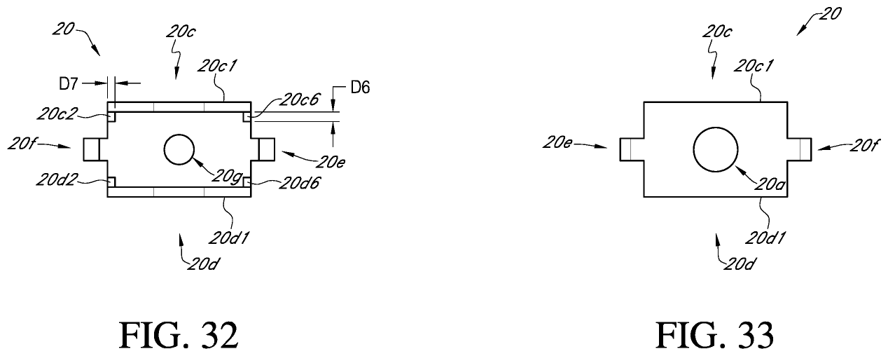

Turning to FIG. 32, depicted therein is a side-elevational end view of insertion tool 20 of FIG. 28. In implementations, insertion tool 20 is shown to include linear dimension D1, and linear dimension D2.

Turning to FIG. 33, depicted therein is a side-elevational end view of insertion tool 20 of FIG. 28.

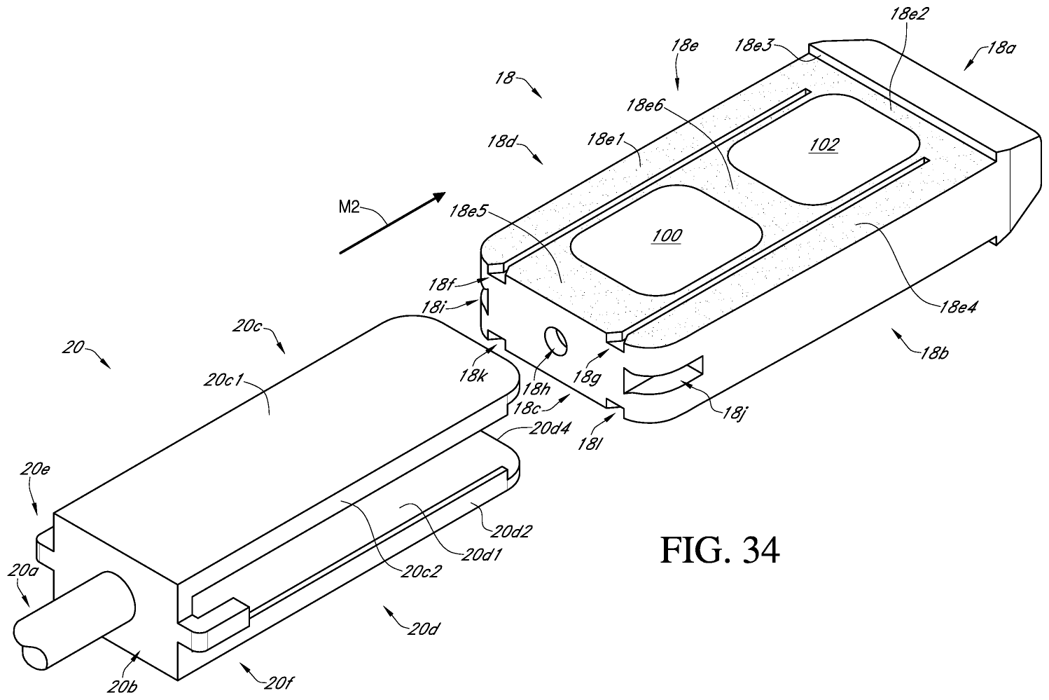

Turning to FIG. 34, depicted therein is a perspective view of interbody spacer 18 of FIG. 24 and insertion tool 20 of FIG. 28 with insertion tool 20 moving direction M2 toward interbody spacer 18 before engagement therewith.

Surfaces of left upper channel 18f, right upper channel 18g, left lower channel 18k, right lower channel 18l shown in FIGS. 24, 26, and 34 to be generally parallel with surfaces such as left upper surface 18e1, right upper surface 18e4, left lower surface 18o1, and right lower surface 18o4 in some implementations can include at least one protrusion, depression, or both (not shown).

Surfaces of right side 20c2, left side 20c6, right side 20d2, left side 20d6 shown in FIGS. 28, 29, 31, 32, and 34 to be generally parallel with surfaces such as exterior surface 20c1, and interior surface 20d1 in some implementations can include at least one protrusion, depression, or both (not shown).

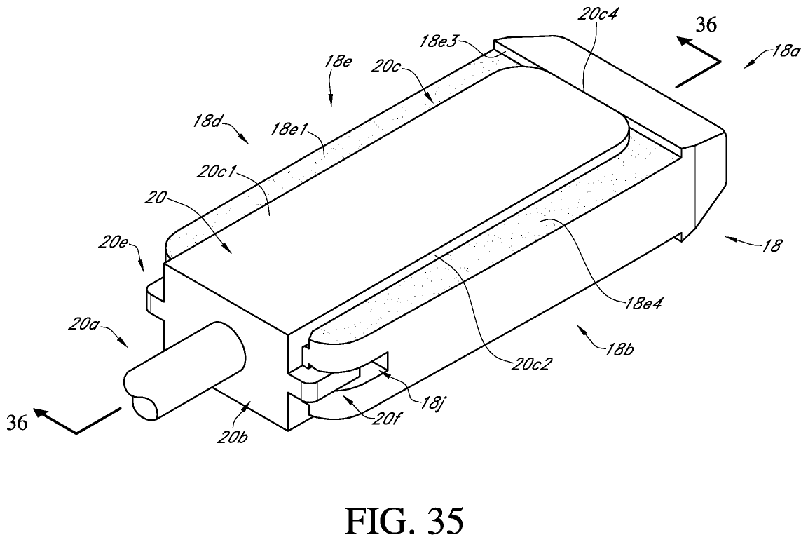

Turning to FIG. 35, depicted therein is a perspective view of interbody spacer 18 of FIG. 24 and insertion tool 20 of FIG. 28 as engaged therewith.

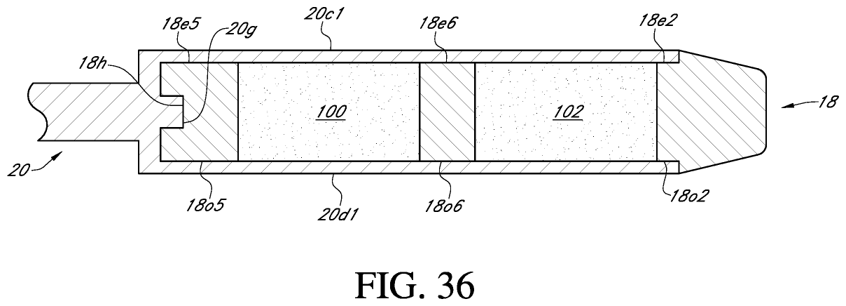

Turning to FIG. 36, depicted therein is a side-elevational cross-sectional view of insertion tool 20 of FIG. 28 engaged with interbody spacer 18 of FIG. 24 taken along cut line 36-36 shown in FIG. 35.

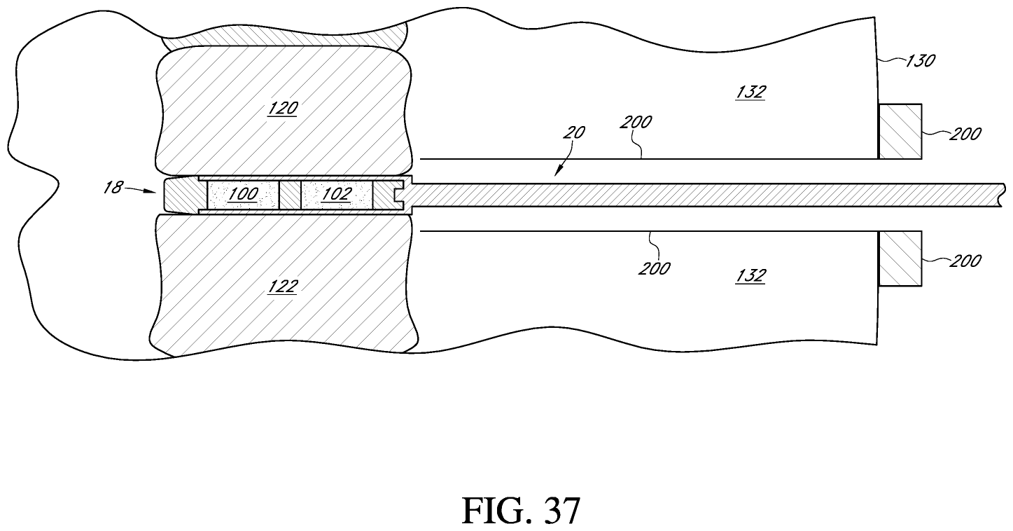

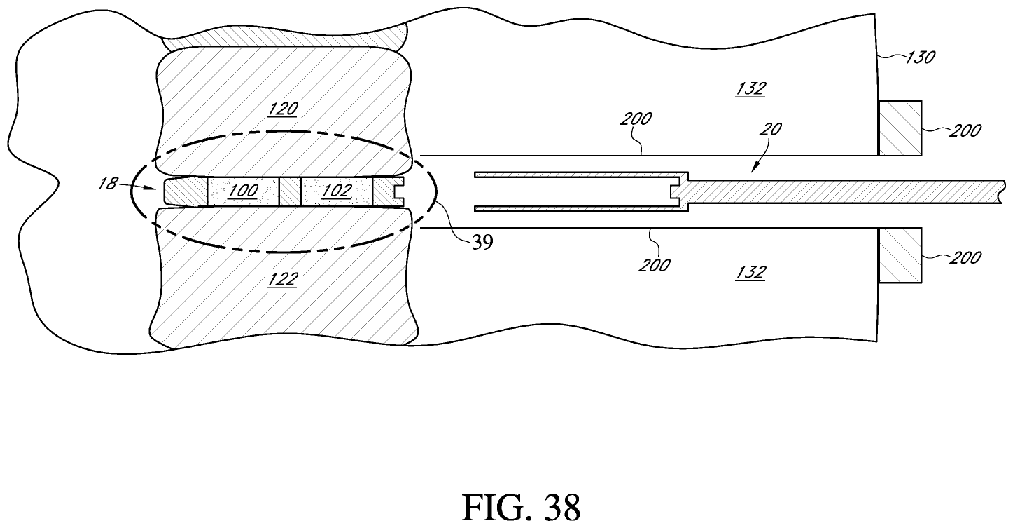

Turning to FIG. 37, depicted therein is a side-elevational cross-sectional view of interbody spacer 18 of FIG. 24 engaged with insertion tool 20 of FIG. 28 fully positioned into a gap between two vertebrae.

Turning to FIG. 38, depicted therein is a side-elevational cross-sectional view of interbody spacer 18 of FIG. 24 fully positioned into a gap between two vertebrae and insertion tool 20 of FIG. 28 being disengaged and retracted therefrom.

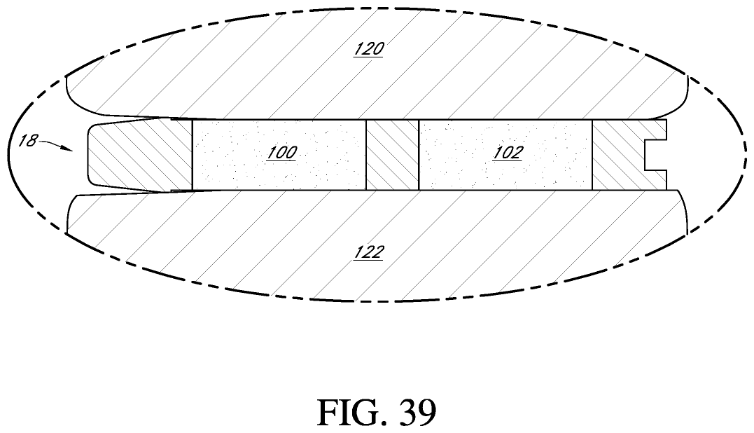

Turning to FIG. 39, depicted therein is an enlarged side-elevational cross-sectional view of a dashed-circle portion of interbody spacer 18 of FIG. 24 positioned in a gap between two vertebrae of FIG. 38 labeled “39” shown in FIG. 38.

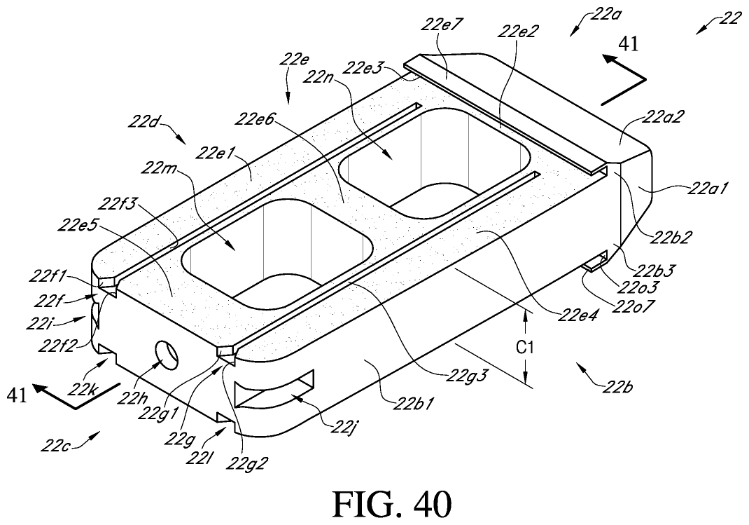

Turning to FIG. 40, depicted therein is a perspective view of interbody spacer 22. In implementations, interbody spacer 22 is shown to include distal tapered lateral end 22a, right side 22b, proximal lateral end 22c, left side 22d, upper side 22e, left upper channel 22f, right upper channel 22g, receptacle opening 22h, left groove 22i right groove 22j, left lower channel 22k, right lower channel 22l, proximal cavity 22m, distal cavity 22n, distal lower edge 22o3, and lower lip portion 22o7. In implementations, distal tapered lateral end 22a is shown to include right surface 22a1, and upper surface 22a2. In implementations, right side 22b is shown to include right surface portion 22b1, upper distal right surface portion 22b2, and lower distal right surface portion 22b3. In implementations, upper side 22e is shown to include left upper surface 22e1, distal upper surface 22e2, distal upper edge 22e3, right upper surface 22e4, right upper surface 22e5, mid upper surface 22e6, and upper lip portion 22e7. In implementations, left upper channel 22f is shown to include left beveled distal edge 22f1, right beveled distal edge 22f2, and left side 22f3. In implementations, right upper channel 22g is shown to include left beveled distal edge 22g1, right beveled distal edge 22g2, and left side 22g3.

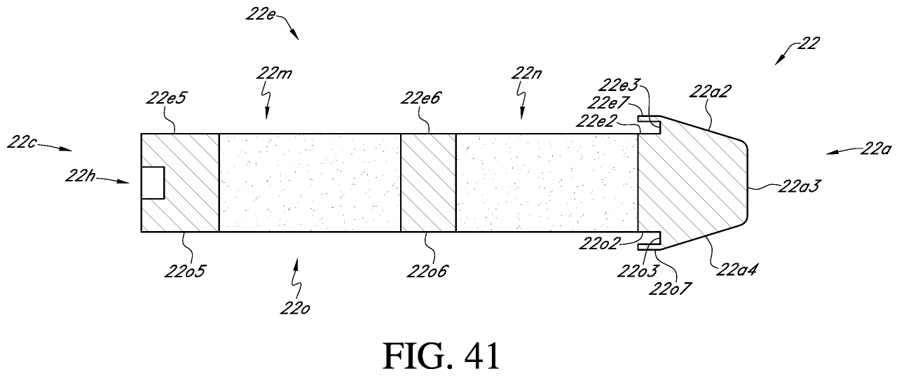

Turning to FIG. 41, depicted therein is a side-elevational cross-sectional view of interbody spacer 22 of FIG. 40 taken along cut line 41-41 shown in FIG. 40. In implementations, interbody spacer 22 is shown to include lower side 22o. In implementations, distal tapered lateral end 22a is shown to include distal surface 22a3, and lower surface 22a4. In implementations, lower side 22o is shown to include distal lower surface 22o2, proximal upper surface 22o5 and mid upper surface 22o6.

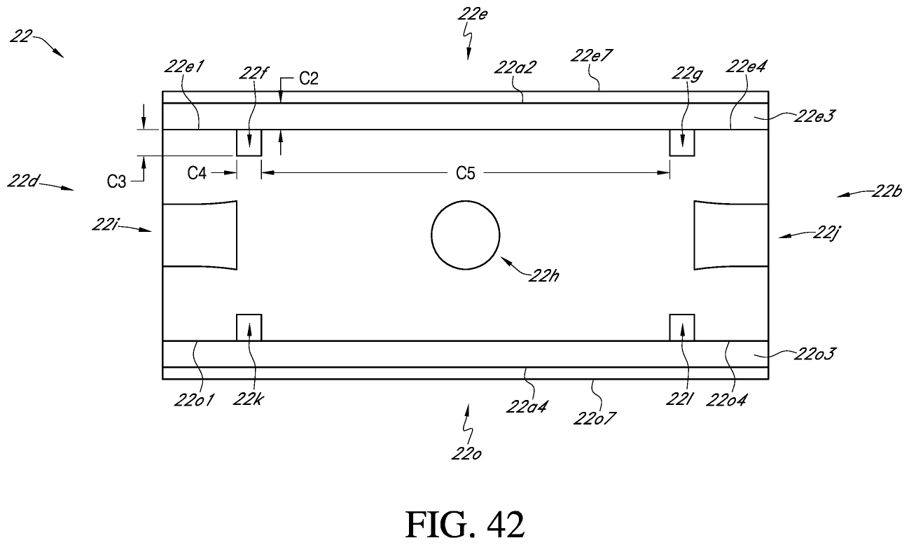

Turning to FIG. 42, depicted therein is a side-elevational end view of interbody spacer 22 of FIG. 41. In implementations, lower side 22o is shown to include left lower surface 22o1, and right lower surface 22o4.

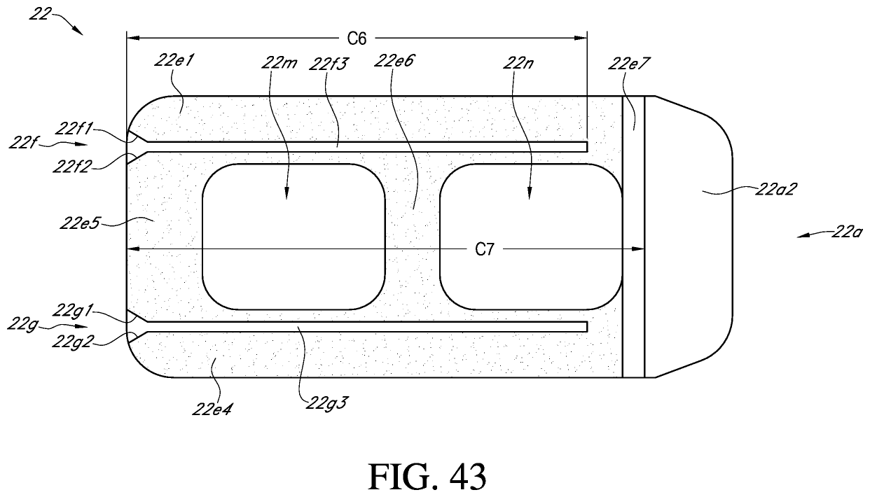

Turning to FIG. 43, depicted therein is a top plan view of interbody spacer 22 of FIG. 40.

Surfaces of left upper chanel 22f, right upper channel 22g, left lower channel 22k, right lower channel 22l shown in FIGS. 40, 42, and 43 to be generally parallel with surfaces such as left upper surface 22e1, right upper surface 22e4, left lower surface 22o1, and right lower surface 22o4 in some implementations can include at least one protrusion, depression, or both (not shown).

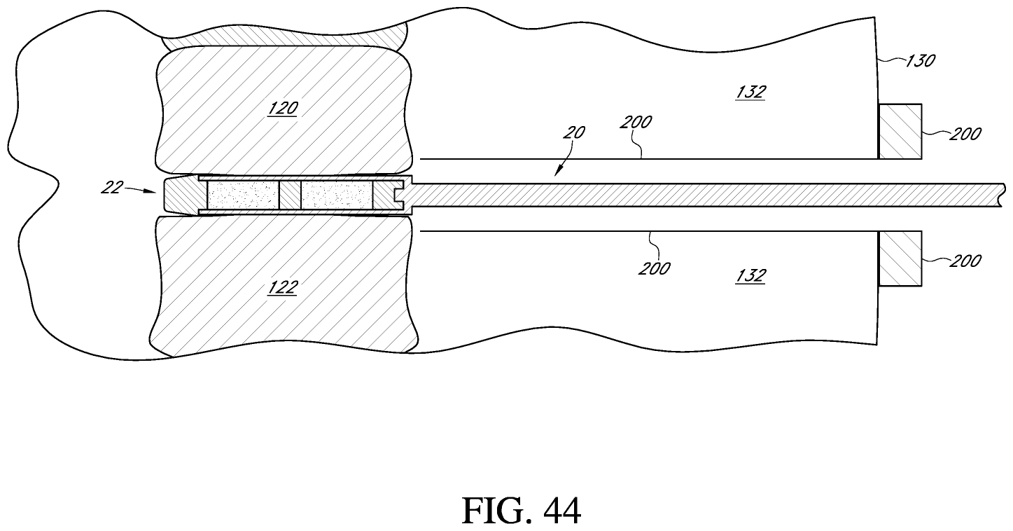

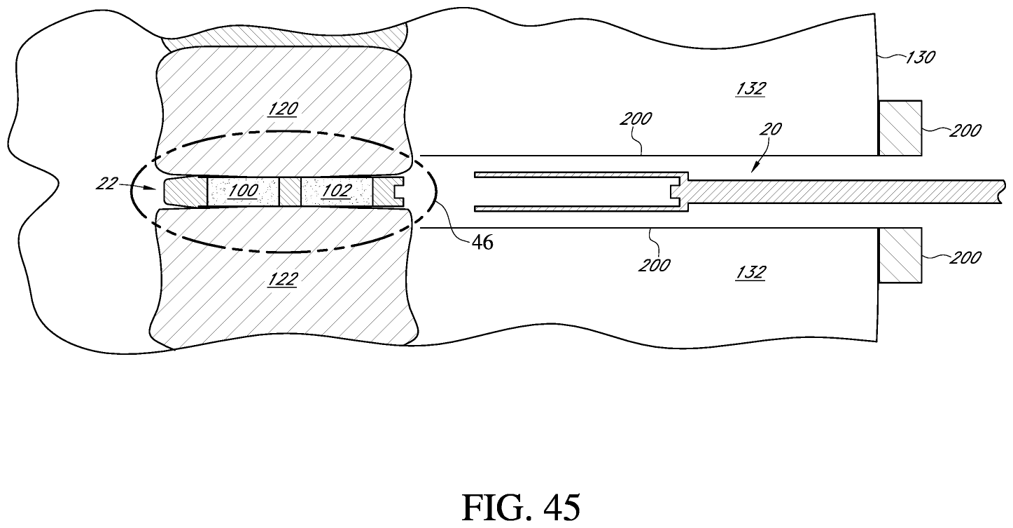

Turning to FIG. 44, depicted therein is a side-elevational cross-sectional view of interbody spacer 22 of FIG. 40 engaged with insertion tool 20 of FIG. 28 fully positioned into a gap between two vertebrae.

Turning to FIG. 45, depicted therein is a side-elevational cross-sectional view of interbody spacer 22 of FIG. 40 fully positioned into a gap between two vertebrae and insertion tool 20 of FIG. 28 being disengaged and retracted therefrom.

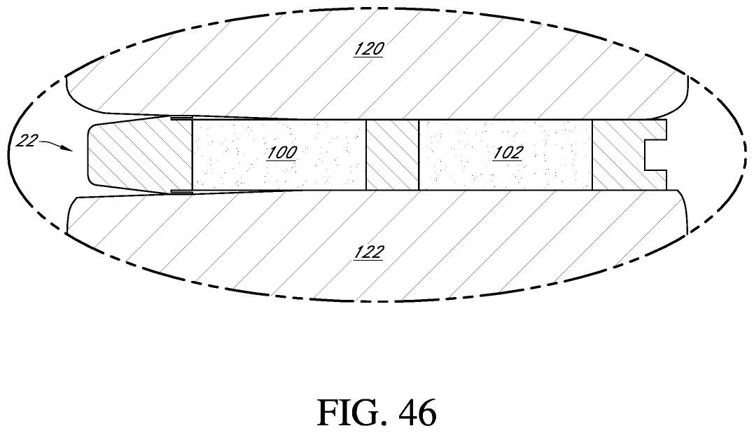

Turning to FIG. 46, depicted therein is an enlarged side-elevational cross-sectional view of a dashed-circle portion of interbody spacer 22 of FIG. 40 positioned in a gap between two vertebrae of FIG. 45 labeled “46” shown in FIG. 45.

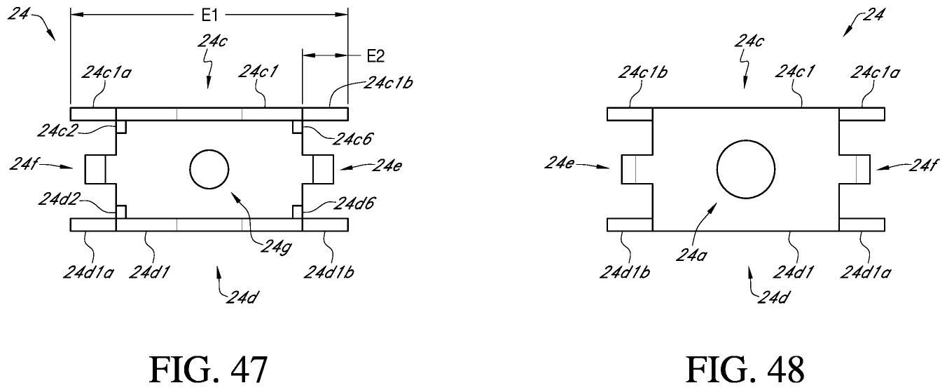

Turning to FIG. 47, depicted therein is a side-elevational end view of insertion tool 24. In implementations, insertion tool 24 is shown to include upper plate member 24c, lower plate member 24d, left tongue member 24e, right tongue member 24f, and engagement member 24g. In implementations, upper plate member 24c is shown to include exterior surface 24c1, right upper wing portion 24c1a, left upper wing portion 24c1b, right side 24c2, and left side 24c6. In implementations, lower plate member 24d is shown to include lower plate member 24d, interior surface 24d1, right upper wing portion 24d1a, left upper wing portion 24d1b, right side 24d2, and left side 24d6.

Surfaces of right side 24c2, left side 24c6, right side 24d2, left side 24d6 shown in FIG. 47 to be generally parallel with surfaces such as 24c1, and 24d1 in some implementations can include at least one protrusion, depression, or both (not shown).

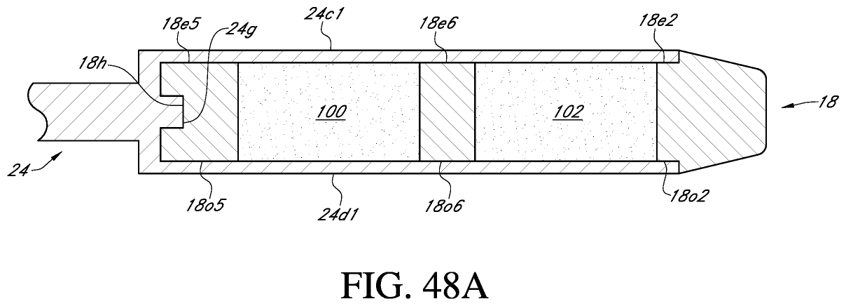

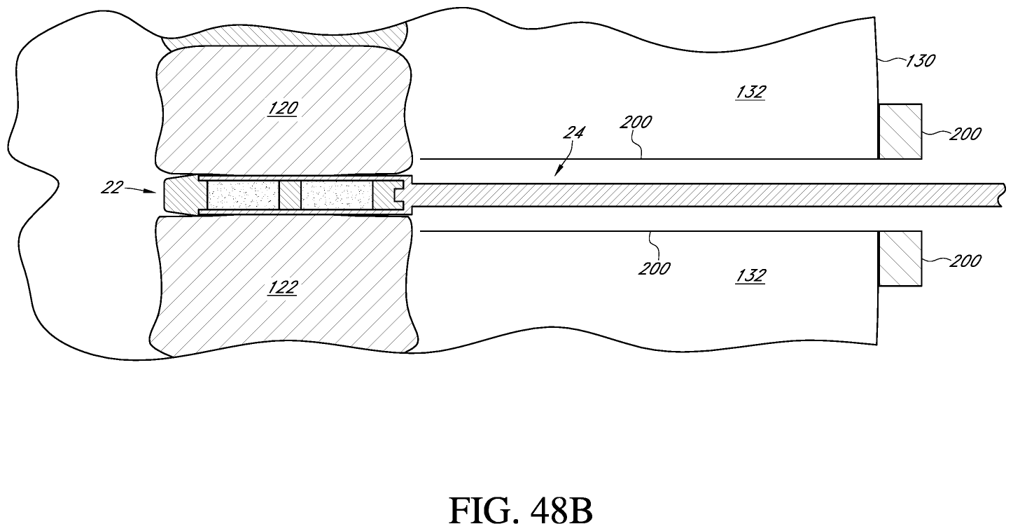

Turning to FIG. 48, depicted therein is a side-elevational end view of insertion tool 24 of FIG. 47. In implementations, insertion tool 24 is shown to include elongated member 24a.

Turning to FIG. 48A, depicted therein is a cross-sectional side-elevational view of insertion tool 24 of FIG. 47.

Turning to FIG. 48B, depicted therein is a side-elevational cross-sectional view of interbody spacer 22 of FIG. 40 engaged with insertion tool 24 of FIG. 47 fully positioned into a gap between two vertebrae.

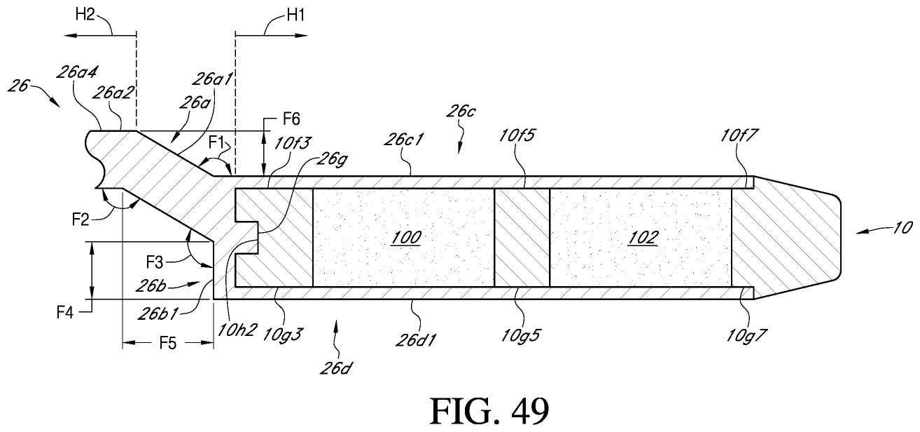

Turning to FIG. 49, depicted therein is a side-elevational cross-sectional view of insertion tool 26. In implementations, insertion tool 26 is shown to include elongated member 26a, support member 26b, upper plate member 26c, lower plate member 26d, and engagement member 26g. In implementations, elongated member 26a is shown to include distal portion 26a1, proximal portion 26a2, and upper surface 26a3. In implementations, support member 26b is shown to include proximal surface 26b1. In implementations, upper plate member 26c is shown to include exterior surface 26c1. In implementations, lower plate member 26d is shown to include exterior surface 26d1. In implementations, insertion tool 26 is shown to include angular dimension F1, angular dimension F2, angular dimension F3, linear dimension F4, linear dimension F5, linear dimension F6, direction H1, and direction H2.

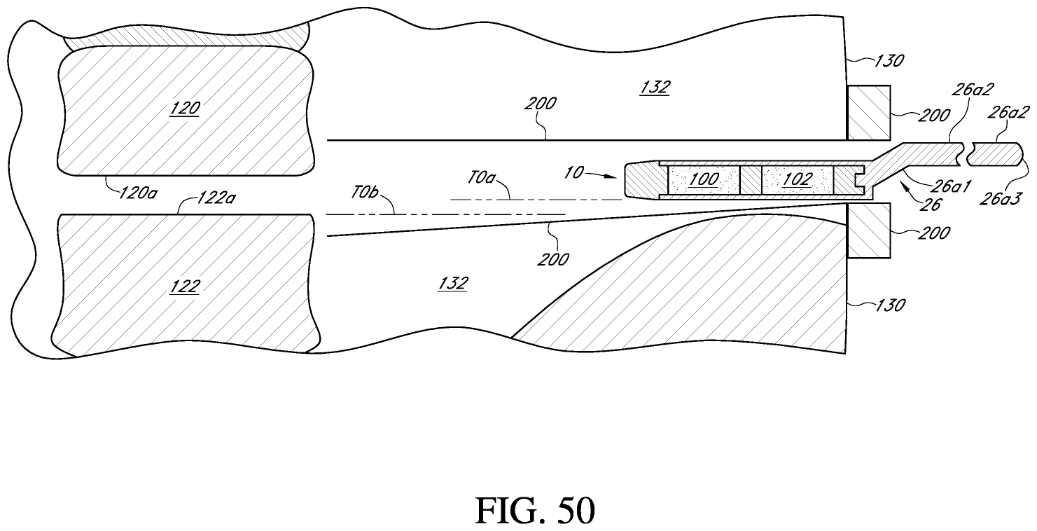

Turning to FIG. 50, depicted therein is a side-elevational cross-sectional view of interbody spacer 10 of FIG. 1 engaged with insertion tool 26 of FIG. 49 at a first angle as described by reference line T0a with respect to reference line T0b and commencing to be positioned into a gap between two vertebrae. In implementations, insertion tool 26 is shown to include upper surface 26a3.

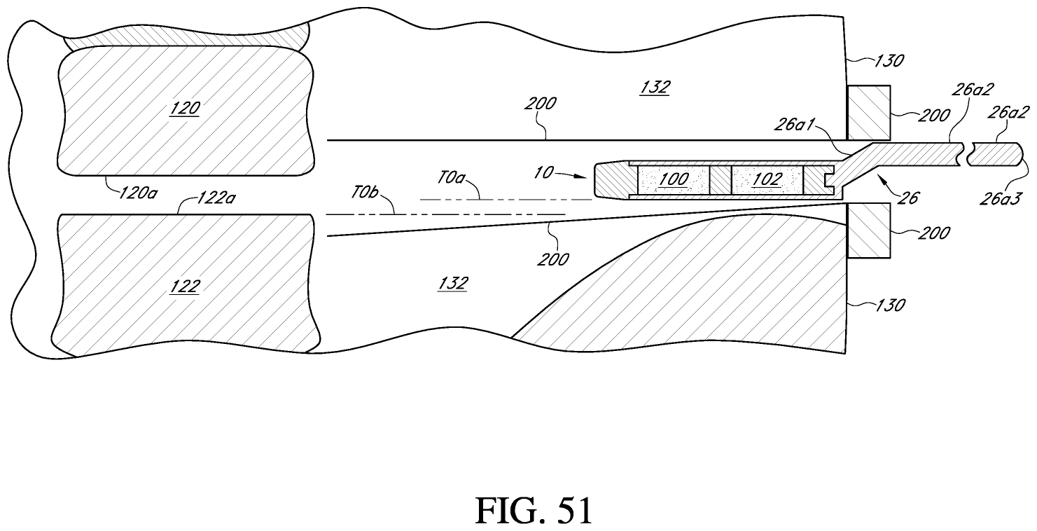

Turning to FIG. 51, depicted therein is a side-elevational cross-sectional view of interbody spacer 10 of FIG. 1 engaged with insertion tool 26 of FIG. 49 at first angle of FIG. 50 and incrementally closer to be positioned into a gap between two vertebrae.

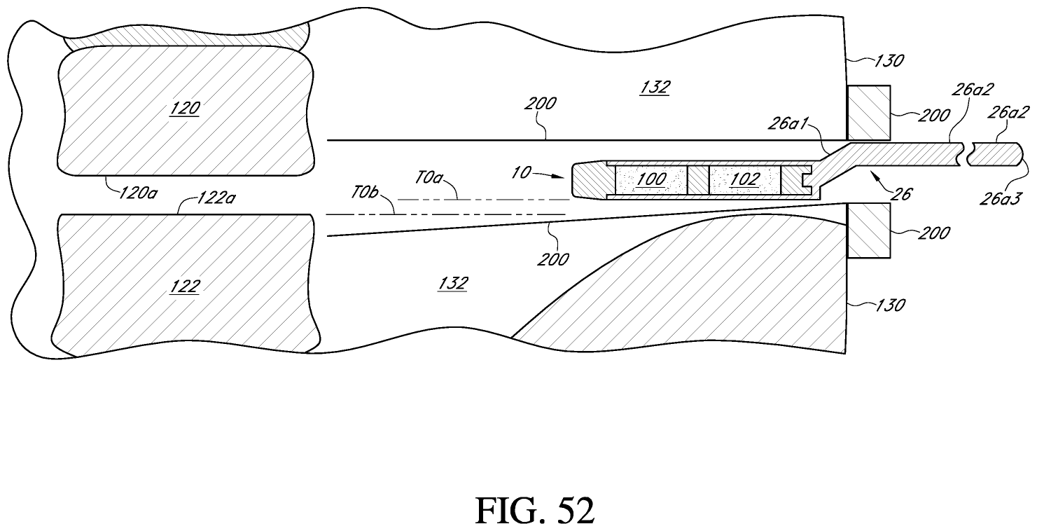

Turning to FIG. 52, depicted therein is a side-elevational cross-sectional view of interbody spacer 10 of FIG. 1 engaged with insertion tool 26 of FIG. 49 at a first angle of FIG. 50 and incrementally closer to be positioned into a gap between two vertebrae.

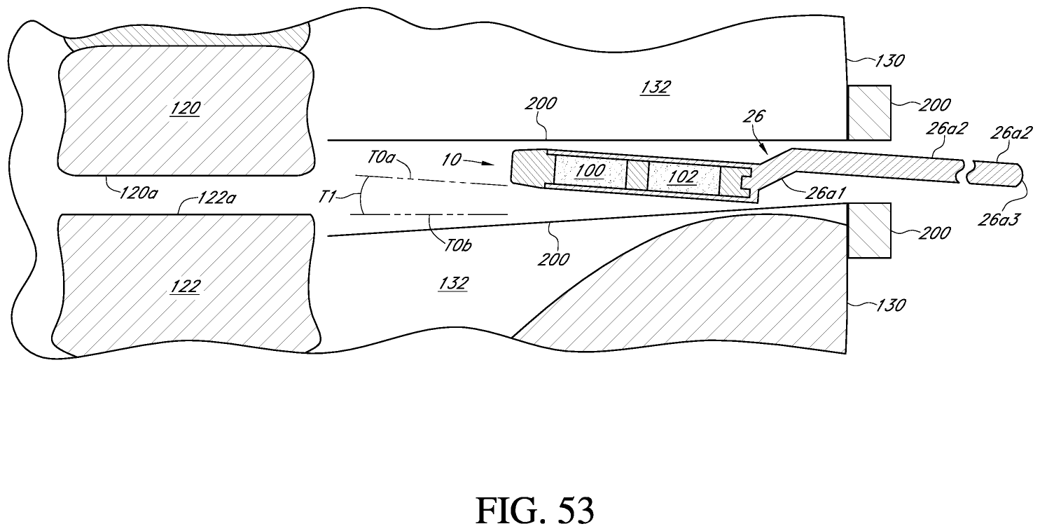

Turning to FIG. 53, depicted therein is a side-elevational cross-sectional view of interbody spacer 10 of FIG. 1 engaged with insertion tool 26 of FIG. 49 at a second angle and incrementally closer to be positioned into a gap between two vertebrae.

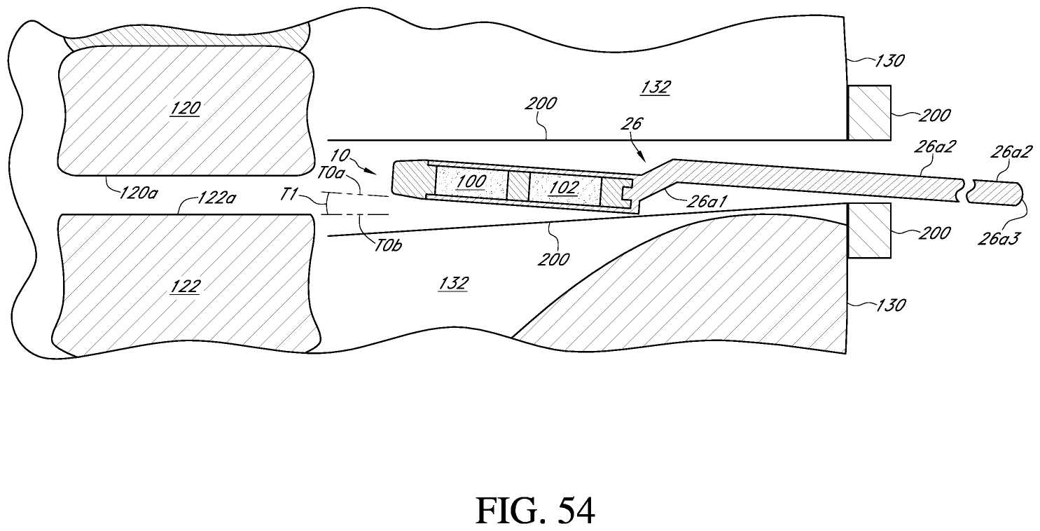

Turning to FIG. 54, depicted therein is a side-elevational cross-sectional view of interbody spacer 10 of FIG. 1 engaged with insertion tool 26 of FIG. 49 at second angle of 53 and incrementally closer to be positioned into a gap between two vertebrae.

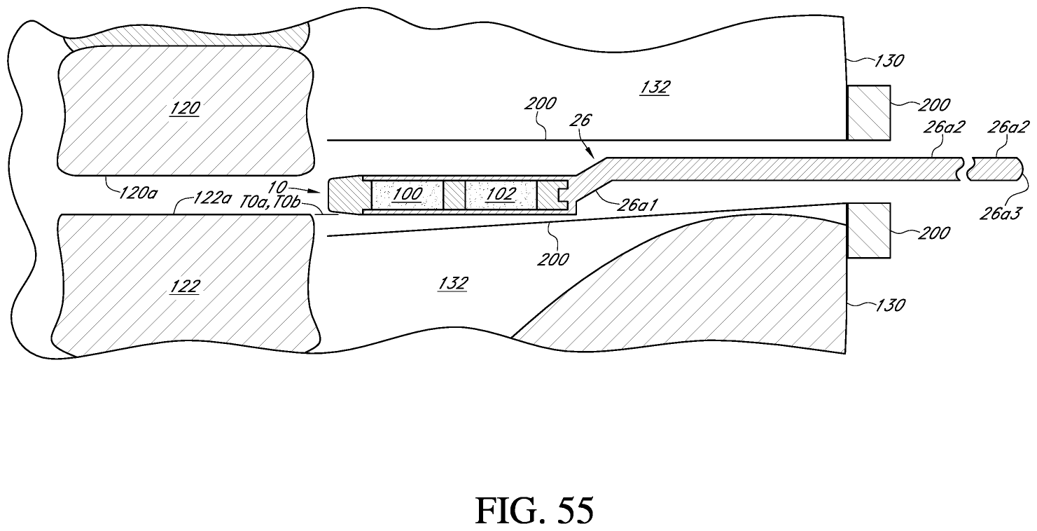

Turning to FIG. 55, depicted therein is a side-elevational cross-sectional view of interbody spacer 10 of FIG. 1 engaged with insertion tool 26 of FIG. 49 at first angle of FIG. 50 and incrementally closer to be positioned into a gap between two vertebrae.

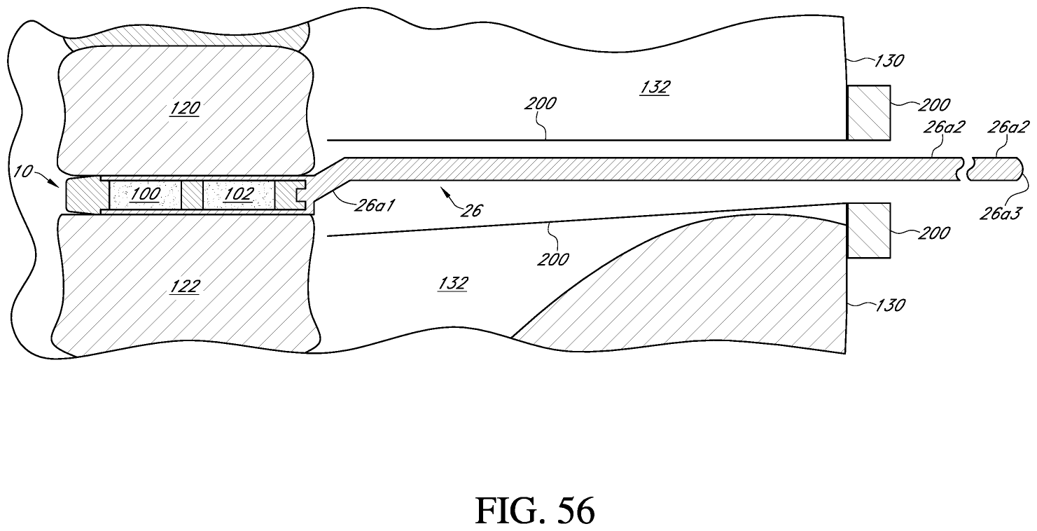

Turning to FIG. 56, depicted therein is a side-elevational cross-sectional view of interbody spacer 10 of FIG. 1 engaged with insertion tool 26 of FIG. 49 fully positioned into a gap between two vertebrae.

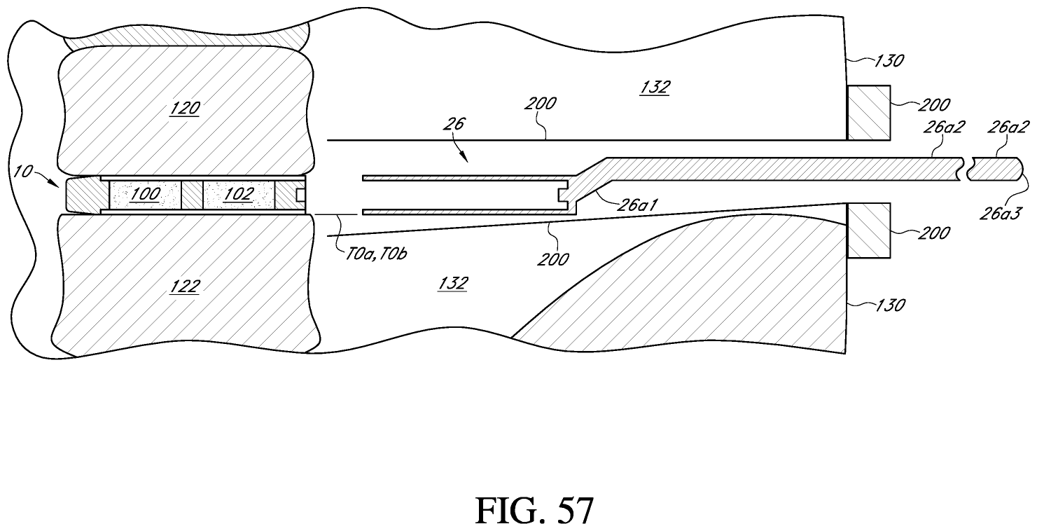

Turning to FIG. 57, depicted therein is a side-elevational cross-sectional view of interbody spacer 10 of FIG. 1 fully positioned into a gap between two vertebrae and insertion tool 26 of FIG. 49 at first angle of FIG. 50 being disengaged and retracted therefrom.

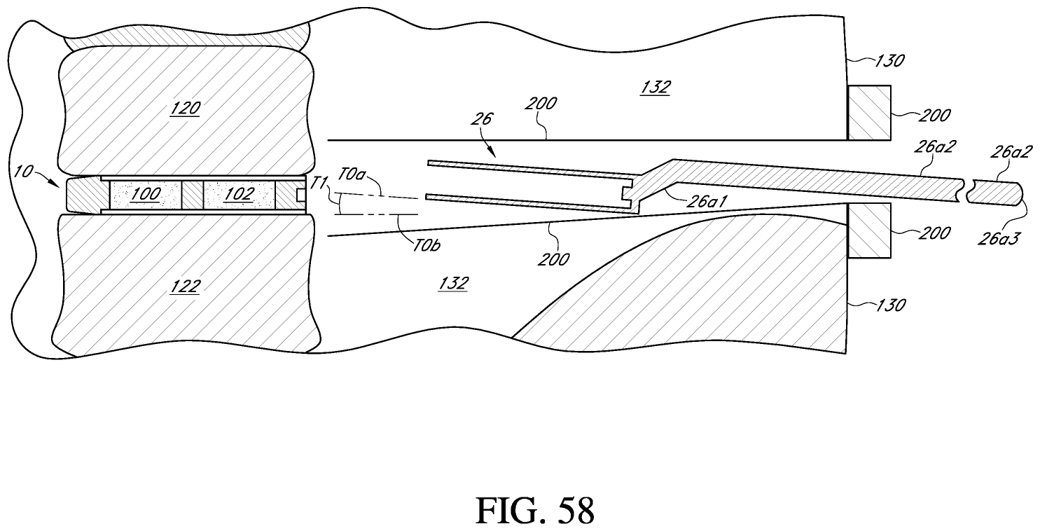

Turning to FIG. 58, depicted therein is a side-elevational cross-sectional view of interbody spacer 10 of FIG. 1 fully positioned into a gap between two vertebrae and insertion tool 26 of FIG. 49 at second angle of FIG. 53 being disengaged and further incrementally retracted therefrom.

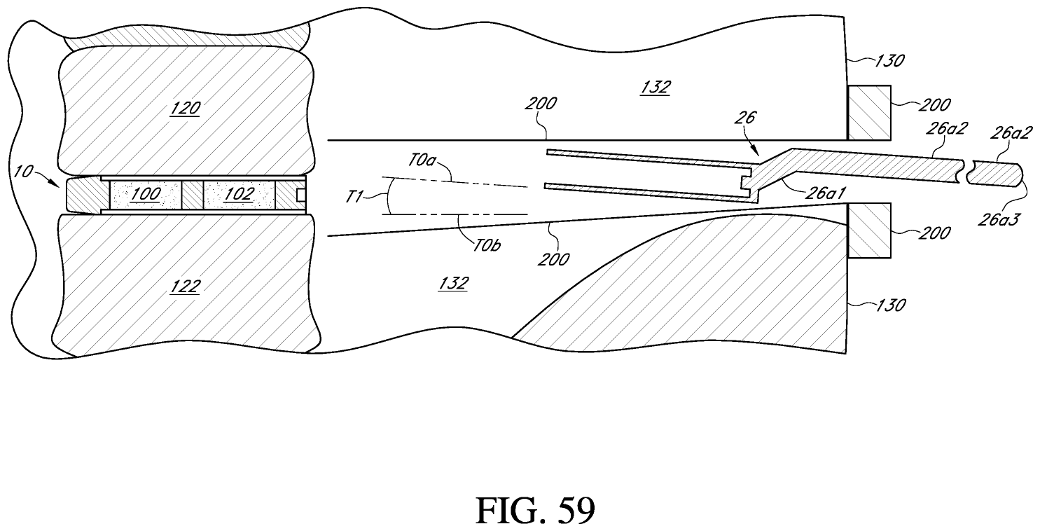

Turning to FIG. 59, depicted therein is a side-elevational cross-sectional view of interbody spacer 10 of FIG. 1 fully positioned into a gap between two vertebrae and insertion tool 26 of FIG. 49 at second angle of FIG. 53 being disengaged and further incrementally retracted therefrom.

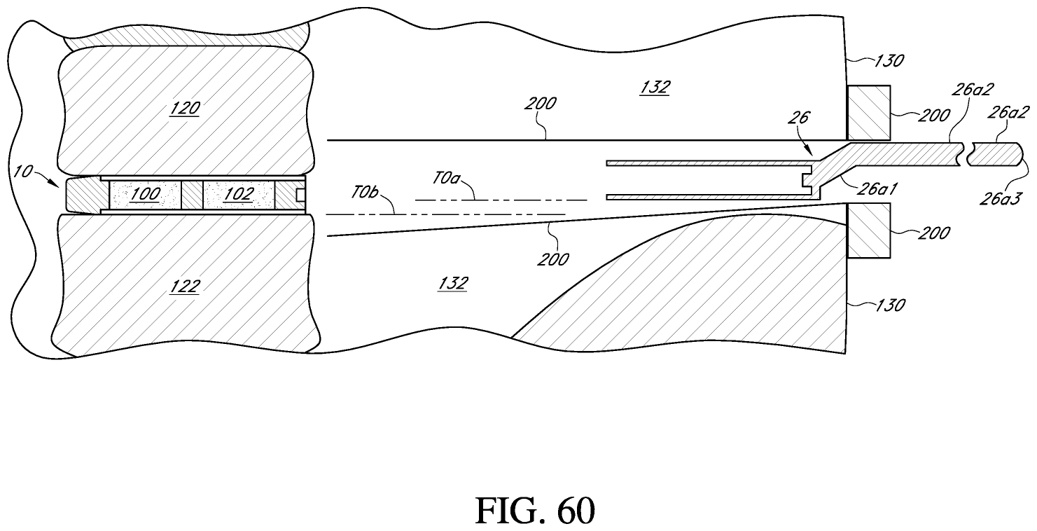

Turning to FIG. 60, depicted therein is a side-elevational cross-sectional view of interbody spacer 10 of FIG. 1 fully positioned into a gap between two vertebrae and insertion tool 26 of FIG. 49 at first angle of FIG. 50 being disengaged and further incrementally retracted therefrom.

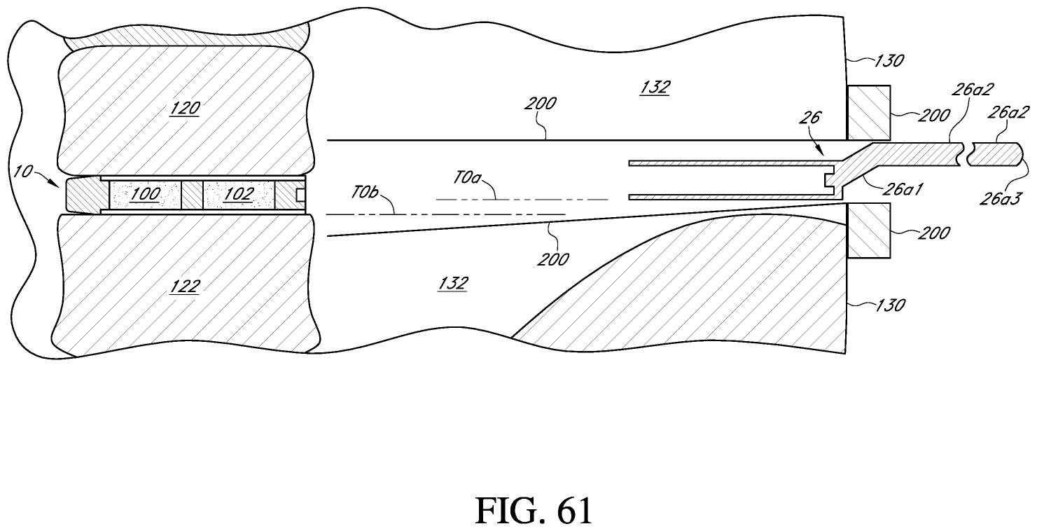

Turning to FIG. 61, depicted therein is a side-elevational cross-sectional view of interbody spacer 10 of FIG. 1 fully positioned into a gap between two vertebrae and insertion tool 26 of FIG. 49 at first angle of FIG. 50 being disengaged and further incrementally retracted therefrom.

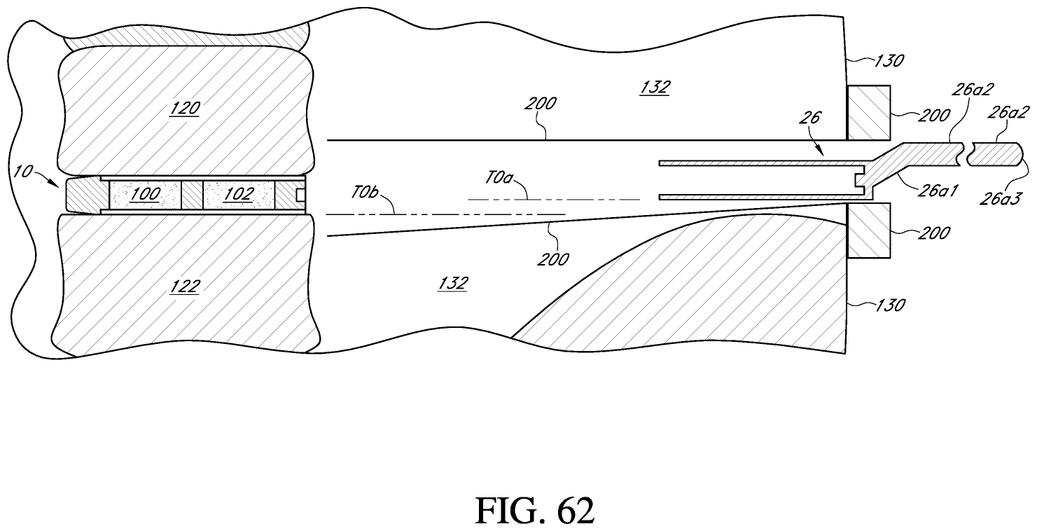

Turning to FIG. 62, depicted therein is a side-elevational cross-sectional view of interbody spacer 10 of FIG. 1 fully positioned into a gap between two vertebrae and insertion tool 26 of FIG. 49 at first angle of FIG. 50 being disengaged and further incrementally retracted therefrom.

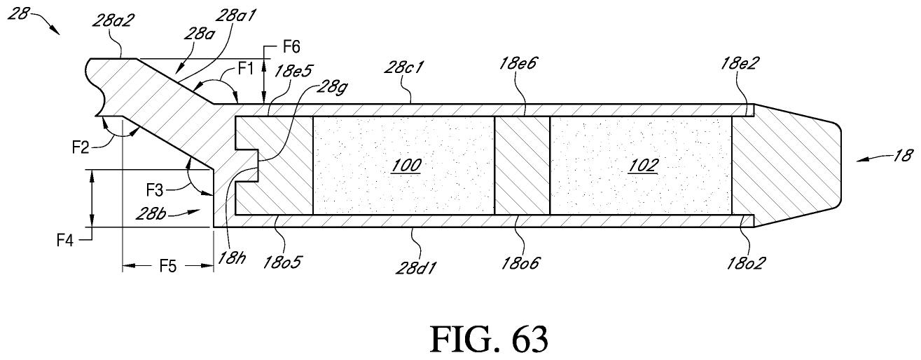

Turning to FIG. 63, depicted therein is a side-elevational cross-sectional view of interbody spacer 18 of FIG. 24 being engaged with insertion tool 28. In implementations, insertion tool 28 is shown to include elongated member 28a, support member 28b, exterior surface 28c1, interior surface 28d1, and engagement member 28g. In implementations, elongated member 28a is shown to include distal portion 28a1, and proximal portion 28a2.

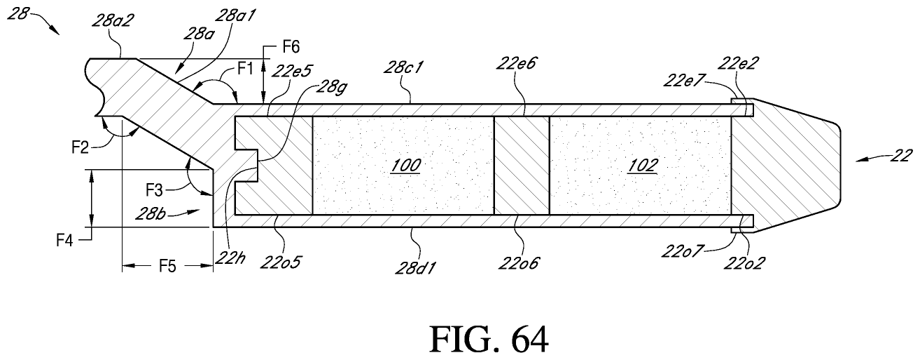

Turning to FIG. 64, depicted therein is a side-elevational cross-sectional view of interbody spacer 22 of FIG. 40 being engaged with insertion tool 28.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. A system for bone graft material, the system comprising:
(I) an interbody spacer including:
(A) at least one side, and
(B) at least one cavity for containing the bone graft material, and;
(II) an insertion tool,
wherein the at least one cavity being inaccessible from the at least one side when the insertion tool is engaged with the interbody spacer, and
wherein the at least one cavity being accessible from the at least one side when the insertion tool is disengaged from the interbody spacer.

2. The system of claim 1 wherein
(A) the at least one side of the interbody spacer includes at least one depression, and
(B) the insertion tool includes at least one shield sized to block accessibility to the at least one cavity from the at least one side of the interbody spacer when the insertion tool is engaged with the interbody spacer, and
(C) the insertion tool being slideably engageable and slideably disengageable with the at least one depression of the interbody spacer.

3. The system of claim 2 wherein the at least one depression of the interbody spacer being sized and shaped to receive the at least one shield of the insertion tool when the at least insertion tool is engaged with the interbody spacer.

4. The system of claim 3 wherein the at least one depression includes at least one sunken planar surface.

5. The system of claim 3 wherein the at least one shield of the insertion tool includes at least one plate member, the at least one plate member being slideably engageable and slideably disengageable with the at least one depression of the interbody spacer.

6. The system of claim 5 wherein the at least one plate member of the insertion tool being generally rectangular in shape.

7. The system of claim 6 wherein the at least one plate member of the insertion tool including a thickness of less than 3 mm.

8. The system of claim 6 wherein the at least one plate member of the insertion tool being fabricated from stainless steel.

9. The system of claim 2 wherein
(A) the at least one depression of the interbody spacer includes at least one channel,
(B) the at least one plate member of the insertion tool includes a planar surface and at least one side extending from the planar surface, and
(C) the at least one channel of the interbody spacer being sized to receive the at least one side of the at least one plate member when the planar surface of the at least one plate member is in contact with the interbody spacer.

10. The system of claim 9 wherein the at least one channel includes a beveled open end.

11. The system of claim 9 wherein the at least one channel includes a first channel and a second channel having the at least one cavity positioned therebetween.

12. The system of claim 9 wherein
(A) the at least one side of the interbody spacer includes
(1) at least one planar surface portion, and
(2) an edge surface extending perpendicularly to the at least one planar surface portion,
(B) the at least one plate member of the insertion tool includes an end, and
(C) the edge surface of the interbody spacer being in contact with the end of the at least one plate member when the insertion tool is engaged with the interbody spacer.

13. The system of claim 12 wherein the at least one channel includes an end, the end being adjacent to the edge surface.

14. The system of claim 12 wherein the at least one side includes a lip member extending from the edge surface, the lip member being parallel with the at least one planar surface portion and spaced from the at least one planar surface portion to form a gap therebetween.

15. The system of claim 9 wherein
(A) the at least one depression of the interbody spacer includes at least one channel,
(B) the at least one plate member of the insertion tool includes a planar surface and at least one side extending from a portion of the planar surface located other than on an edge of the planar surface, and
(C) the at least one channel of the interbody spacer being sized to receive the at least one side of the at least one plate member when the planar surface of the at least one plate member is in contact with the interbody spacer.

16. The system of claim 1 wherein the insertion tool includes
(A) a support member,
(B) at least one plate member having an exterior surface extending in a first direction away from the support member,
(C) an elongated member having a first portion and a second portion, the first portion extending from the support member, the second portion extending from the first portion in a second direction opposite the first direction, and
(D) the support member includes a rear surface, the first portion of the elongated member extending other than perpendicularly from the rear surface of the support member.

17. The system of claim 1 wherein the insertion tool includes
(A) a support member,
(B) at least one plate member having an exterior surface extending in a first direction away from the support member,
(C) an elongated member having a first portion and a second portion, the first portion extending from the support member, the second portion extending from the first portion in a second direction opposite the first direction, and
(D) the support member includes a rear surface, the first portion of the elongated member extending from the rear surface of the support member other than parallel with the first and second directions.

18. The system of claim 1 wherein the insertion tool includes
(A) a support member,
(B) at least one plate member having an exterior surface extending in a first direction away from the support member,
(C) an elongated member having a first portion and a second portion, the first portion extending from the support member, the second portion extending from the first portion in a second direction opposite the first direction, and
(D) the second portion of the elongated member including an upper surface being positioned in a first plane that is unoccupied by any other portion of the insertion tool, the first plane being spaced from the exterior surface of the at least one plate member a first distance.

19. (canceled)

20. An interbody spacer for bone graft material, the interbody spacer comprising:
(I) at least one side including:
(A) at least one planar surface portion,
(B) at least one cavity, the at least one cavity for containing the bone graft material,
(C) at least one opening into the at least one cavity, the at least one opening being adjacent the at least one planar surface portion,
(D) a first channel extending into other portions of the interbody spacer from the at least one planar surface portion, and
(E) a second channel extending into other portions of the interbody spacer from the at least one planar surface portion,
wherein the at least one opening into the at least one cavity being positioned between the first channel and the second channel.

21. An insertion tool for an interbody spacer, the insertion tool comprising:
(I) a support member;
(II) an elongated member, at least a portion of the elongated member extending from the support member along a first direction; and
(III) at least one plate extending from the support member along a second direction, the second direction being opposite the first direction.

22. (canceled)

23. (canceled)

24. An interbody spacer comprising:
(I) at least one planar surface portion, and
(II) an edge surface extending perpendicularly to the at least one planar surface portion.

25. The interbody spacer of claim 24 further including a lip member extending from the edge surface, the lip member being parallel with the at least one planar surface portion and spaced from the at least one planar surface portion to form a gap therebetween.

26. (canceled)

27. (canceled)
